(19) United States
(12) Patent Application Publication
(10) Pub. No.: US 2018/0111889 A1
(43) Pub. Date: Apr. 26, 2018

(54) METHODS OF MAKING MULTICYCLIC COMPOUNDS USING MULTICOMPONENT/TANDEM REACTION SEQUENCES

(71) Applicant: Board of Regents of the Nevada System of Higher Education, on Behalf of the University of Nevada,, Reno, NV (US)

(72) Inventors: Wesley A. Chalifoux, Reno, NV (US); Rachael A. Carmichael, Reno, NV (US)

(73) Assignee: Board of Regents of the Nevada System of Higher Education, on Behalf of the University of Nevada,, Reno, NV (US)

(21) Appl. No.: 15/558,981

(22) PCT Filed: Mar. 7, 2016
(86) PCT No.: PCT/US2016/022802

§ 371 Date: Sep. 15, 2017

Related U.S. Application Data
(60) Provisional application No. 62/136,189, filed on Mar. 20, 2015.

(51) Int. Cl. C07C 45/61

(52) U.S. Cl. CPC C07C 45/61; C07C 2603/18; C07C 2602/28

(57) ABSTRACT

Disclosed herein are embodiments of multicyclic compounds and methods of making such compounds. The disclosed methods reduce step-counts in the synthesis of complex targets, while reducing costs and waste streams.

### CROSS REFERENCE TO RELATED APPLICATION

This application claims the benefit of, and priority to, the earlier filing date of U.S. Provisional Patent Application No. 62/136,189, filed on Mar. 20, 2015, which is herein incorporated by reference in its entirety.

### FIELD

Disclosed herein are embodiments of multicyclic compounds and methods for making such multicyclic compounds.

### BACKGROUND

Hirsutellones, as well as other molecules containing a 6-5-6 ring system, are an important class of molecules. Efficient and cost effective syntheses employing step-economic strategies to generate desirable 6-5-6 frameworks are needed for pharmaceutical applications. Progress has been made in designing more advanced cyclic methodology from relatively simple starting materials; however, polycyclization reactions remain a vital area of research due to the challenges of synthetic design.

One of the drawbacks in synthesizing complex polycyclic frameworks is the high step counts, making their use as viable drug targets limited. The improvement of synthetic methods to allow access to complex, biologically active molecules in an efficient manner is therefore an important area of research. Particularly, cis-decalin structures that contain a quaternary or vicinal quaternary centers are seen in a number of sequiterpenoid natural products that have been of synthetic interest. The utilization of alkynes as high energy synthons for the synthesis of useful cyclic and polycyclic intermediates is still an underdeveloped area. Although the use of alkynes as dienophiles is known, their utilization has been fairly limited.

### SUMMARY

Disclosed herein are embodiments of methods of making multicyclic compounds comprising combining an alkyne-containing compound comprising a carbonyl functional group with a diene and a Lewis acid to form a multicyclic compound comprising one or more fused ring systems. In some embodiments, the alkyne-containing compound can be a diynone. The diynone can comprise two terminal functional groups that facilitate regiochemical control. In some embodiments, the two terminal functional groups are selected from aryl, aliphatic, heteroaryl, heteroaliphatic, or —Si(R)₃, wherein each R independently is selected from hydrogen, aliphatic, heteroaliphatic, aryl, or heteroaryl. In particular disclosed embodiments, the diynone can have a structure as disclosed herein. In other embodiments, the alkyne-containing compound can have the formula below.

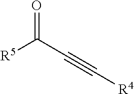

With reference to this formula, R⁴ is selected from hydrogen, aliphatic, aryl, heteroaliphatic, or —Si(R)₃; and R⁵ is selected from aryl, heteroaryl, aliphatic, ester, or carboxylic acid. Exemplary embodiments of such alkyne-containing compounds are disclosed herein.

The diene used in the disclosed methods can have a formula illustrated below.

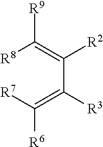

With reference to this diene formula, R² is selected from hydrogen, aliphatic, heteroaliphatic, aryl, or heteroaryl; R³ is selected from hydrogen, aliphatic, heteroaliphatic, aryl, or heteroaryl; and each R⁶, R⁷, R⁸, and R⁹ independently is selected from hydrogen, aliphatic, heteroaliphatic, aryl, or heteroaryl. Exemplary dienes are disclosed herein.

In some embodiments, the Lewis acid can be selected from ZnCl₂, BF₃.Et₂O, SnCl₄, TiCl₄, FeCl₃, AlCl₃, EtAlCl₂, Me₂AlCl, BCl₃, or In(OTf)₃.

In some embodiments, the method can further comprise forming an intermediate having the formula illustrated below.

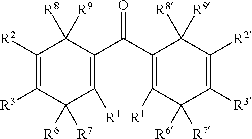

With reference to this formula, each R¹ independently can be selected from aliphatic, aryl, heteroaryl, heteroaliphatic, or —Si(R)₃, wherein each R is selected from hydrogen, aliphatic, heteroaliphatic, aryl, heteroaryl; each R² and R^{2′} independently can be selected from hydrogen, aliphatic, heteroaliphatic, aryl, or heteroaryl; each R³ and R^{3′} independently can be selected from hydrogen, aliphatic, heteroaliphatic, aryl, or heteroaryl; and each R⁶, R⁷, R⁸, R⁹, R^{6′}, R^{7′}, R^{8′}, and R^{9′} independently can be selected from hydrogen, aliphatic, heteroaliphatic, aryl, or heteroaryl; or R⁷ or R^{7′} forms a 5-membered or 6-membered ring with R⁸ or R^{8′}, respectively. Exemplary embodiments of the multicyclic compounds made according to the method also are disclosed herein.

The foregoing and other features and advantages will become more apparent from the following detailed description and figures.

### BRIEF DESCRIPTION OF THE DRAWINGS

FIG. 1 is a ¹H-NMR spectrum of an exemplary multicyclic compound.

FIG. 2 is a ¹³C-NMR spectrum of an exemplary multicyclic compound.

FIG. 3 is a ¹H-NMR spectrum of an exemplary multicyclic compound.

FIG. 4 is a ¹³C-NMR spectrum of an exemplary multicyclic compound.

FIG. 5 is a ¹H-NMR spectrum of an exemplary multicyclic compound.

FIG. 6 is a ¹³C-NMR spectrum of an exemplary multicyclic compound.

FIG. 7 is a ¹H-NMR spectrum of an exemplary multicyclic compound.

FIG. 8 is a ¹³C-NMR spectrum of an exemplary multicyclic compound.

FIG. 9 is a ¹H-NMR spectrum of an exemplary multicyclic compound.

FIG. 10 is a ¹³C-NMR spectrum of an exemplary multicyclic compound.

FIG. 11 is a ¹H-NMR spectrum of an exemplary multicyclic compound.

FIG. 12 is a ¹³C-NMR spectrum of an exemplary multicyclic compound.

FIG. 13 is a ¹H-NMR spectrum of an exemplary multicyclic compound.

FIG. 14 is a ¹H-NMR spectrum of an exemplary multicyclic compound.

FIG. 15 is a ¹H-NMR spectrum of an exemplary multicyclic compound.

FIG. 16 is a NOESY spectrum of an exemplary multicyclic compound.

FIG. 17 is a single crystal X-ray structure image showing a top view of an exemplary multicyclic compound.

FIG. 18 is a single crystal X-ray structure image showing a side view of an exemplary multicyclic compound.

### DETAILED DESCRIPTION

### I. EXPLANATION OF TERMS

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

The disclosed compounds and methods are not limited to any specific aspect or feature or combinations thereof, nor do the disclosed compounds and methods require that any one or more specific advantages be present or problems be solved. Any theories of operation are to facilitate explanation, but the disclosed compounds and methods are not limited to such theories of operation.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” and “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and compounds similar or equivalent to those described herein can be used in the practice or testing of the present disclosure, suitable methods and compounds are described below. The compounds, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

To facilitate review of the various embodiments of the disclosure, the following explanations of specific terms and abbreviations are provided:

Aliphatic: A hydrocarbon, or a radical thereof, having at least one carbon atom to 50 carbon atoms, such as one to 25 carbon atoms, or one to ten carbon atoms, and which includes alkanes (or alkyl), alkenes (or alkenyl), alkynes (or alkynyl), including cyclic versions thereof, and further including straight- and branched-chain arrangements, and all stereo and position isomers as well.

Aldehyde: —C(O)H.

Alkyl: A saturated monovalent hydrocarbon having at least one carbon atom to 50 carbon atoms, such as one to 25 carbon atoms, or one to ten carbon atoms, wherein the saturated monovalent hydrocarbon can be derived from removing one hydrogen atom from one carbon atom of a parent compound (e.g., alkane). An alkyl group can be branched, straight-chain, or cyclic (e.g., cycloalkyl), and all stereo and position isomers as well.

Alkenyl: An unsaturated monovalent hydrocarbon having at least two carbon atoms 50 carbon atoms, such as two to 25 carbon atoms, or two to ten carbon atoms, and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z), and all stereo and position isomers as well.

Alkynyl: An unsaturated monovalent hydrocarbon having at least two carbon atoms 50 carbon atoms, such as two to 25 carbon atoms, or two to ten carbon atoms and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl), and all stereo and position isomers as well.

Alkoxy: —O-alkyl, with exemplary embodiments including, but not limited to, methoxy, ethoxy, n-propoxy, isopropoxy, n-butoxy, t-butoxy, sec-butoxy, n-pentoxy, and the like.

Aryl: An aromatic carbocyclic group comprising at least five carbon atoms to 15 carbon atoms, such as five to ten carbon atoms, having a single ring or multiple condensed rings, which condensed rings can or may not be aromatic provided that the point of attachment is through an atom of the aromatic carbocyclic group.

Carbonyl: —C(O)—.

Diene: A compound comprising at least two carbon double bonds. In some embodiments, a diene can be conjugated.

Diels-Alder Reaction: A cycloaddition reaction between a compound comprising a diene and a compound that acts as a dienophile (with particular disclosed embodiments comprising at least one alkyne). The compound comprising the diene and the dienophile can be the same compound or two separate compounds. In particular disclosed embodiments, the dienophile is an alkyne-containing compound.

Diynone: An alkyne-containing compound comprising two alkyne groups coupled through a carbonyl group. In exemplary embodiments, a diynone has a structure as shown below, wherein each R¹ independently can be selected from aliphatic, aryl, heteroaliphatic, heteroaryl, or Si(R)₃, wherein each R is selected from hydrogen, aliphatic, heteroaliphatic, aryl, or heteroaryl.

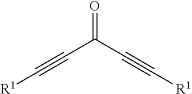

Haloaliphatic: An aliphatic group wherein one or more hydrogen atoms, such as one to 10 hydrogen atoms, independently is replaced with a halogen atom, such as fluoro, bromo, chloro, or iodo.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising, within the group, at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof.

Heteroalkyl/Heteroalkenyl/Heteroalkynyl: An alkyl, alkenyl, or alkynyl group (which can be branched, straight-chain, or cyclic) comprising, within the group, at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof.

Heteroaryl: An aryl group comprising at least one heteroatom to six heteroatoms, such as one to four heteroatoms, which can be selected from, but not limited to oxygen, nitrogen, sulfur, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group.

Lewis Acid: A chemical species that reacts with a Lewis base to form a Lewis adduct, such as a compound or ionic species that can accept an electron pair from a donor compound.

Lewis Base: A chemical species that can donate a pair of electrons to a Lewis acid to form a Lewis adduct, such as a compound or ionic species that can donate an electron pair to an acceptor compound.

Narazov Cyclization: A chemical reaction used to make a five-membered ring system or a multi-cyclic ring system comprising a five-membered ring system.

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein. In embodiments where compounds comprising stereocenters are illustrated, the present disclosure is not limited to the particular enantiomer/diastereomer illustrated and can include racemic mixtures and/or compounds having the opposite stereocenter(s) illustrated by the formulas provided herein.

### II. COMPOUNDS AND METHODS OF MAKING COMPOUNDS

Efficient methods for making polycyclic compounds and intermediates that bear useful handles for further synthetic manipulations are disclosed herein. In some embodiments, the methods comprise utilizing alkyne-containing compounds. The utilization of alkynes as dienophiles in cycloaddition reactions with dienes has many attractive features over traditionally used alkene dienophiles. One example includes the avoidance of extra synthetic steps typically involved in producing pure E or Z alkene products. Additionally, the use of alkynes as dienophiles generates cyclo-1,4-hexadienes (“skipped dienes”), which are otherwise produced by harsh dissolved metal reduction of arenes. Such reduction reactions often require conditions intolerant to sensitive functional groups.

In some embodiments, the development of high-yielding and efficient methods to synthesize complex products from simple starting materials through tandem or domino processes is described. The disclosed methods reduce step-counts in the synthesis of complex targets, while reducing costs and waste streams. In some embodiments, a series of Lewis acid catalyzed tandem cyclization reactions of alkyne starting materials to generate functionalized 6-6-bicyclic and 6-5-6 tricyclic products in a one-pot process in good yields are described. In such embodiments, it is possible to generate 4 to 5 new carbon-carbon bonds, 2 or 3 rings, and vicinal quaternary centers.

In some embodiments, methods of making multicyclic compounds are described wherein the methods can comprise combining an alkyne-containing compound comprising a carbonyl functional group with a diene and a Lewis acid to form a multicyclic compound comprising one or more fused ring systems. A general retrosynthetic route is illustrated below in Scheme 1.

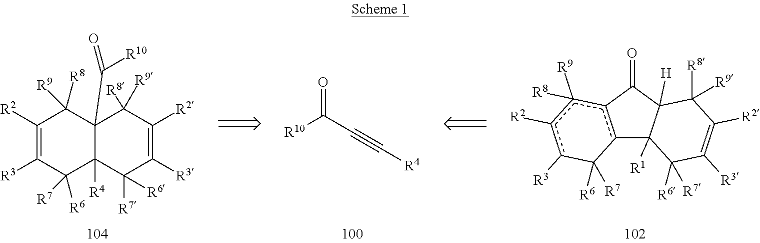

In some embodiments, the methods can comprise using a Lewis Acid catalyzed Diels-Alder/Nazarov cascade reaction yielding a 6-5-6 ring system prevalent in biologically active compounds. Such methods can be used to prepare complex target molecules for pharmaceutical development. In some embodiments, alkyne-containing compounds like 100 can be used in a one-pot synthetic method to produce bicyclic compounds, such as 104 (Scheme 3) and desirable 6-5-6 backbone structures, such as 102, which can be mapped onto an array of biologically active compounds. The methods described herein provide the desired product in good yield with a versatile substrate scope. Exemplary schemes showing additional alkyne-containing compounds and products are illustrated in Schemes 2-4. Additionally, different dienes can be utilized to make asymmetric products, such as compounds 402 and 204′, as illustrated in Scheme 4.

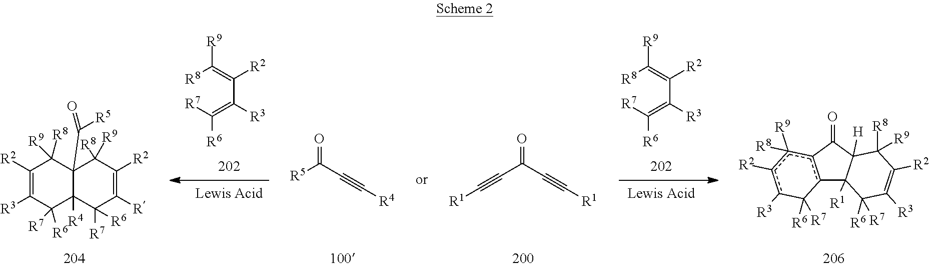

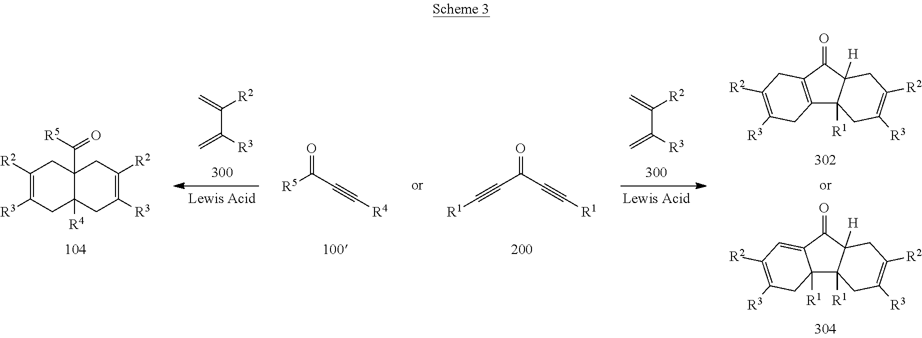

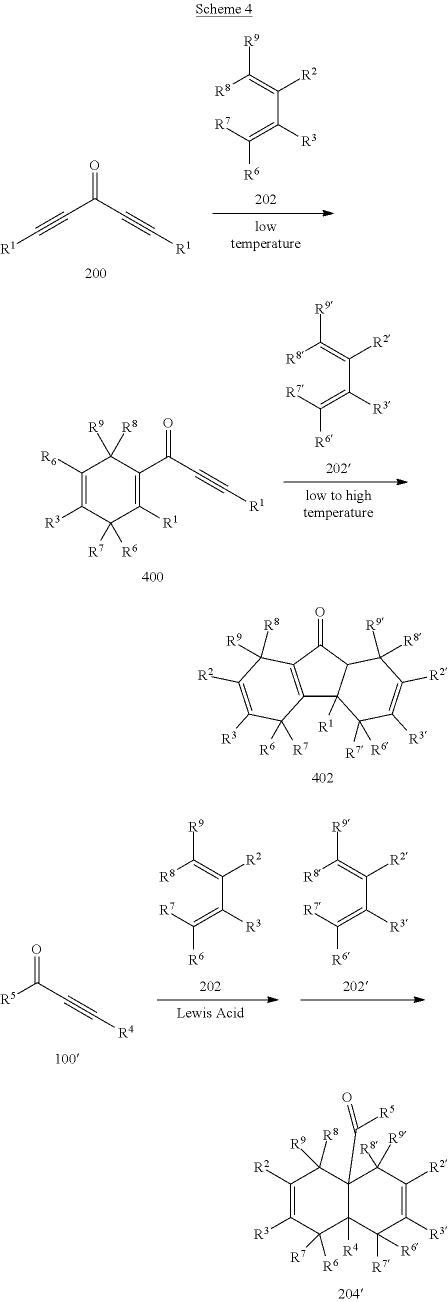

With reference to Schemes 2-4, each R¹ independently can be selected from aliphatic, aryl, heteroaryl, heteroaliphatic, or —Si(R)₃, wherein each R is selected from hydrogen, aliphatic, heteroaliphatic, aryl, or heteroaryl; each R² and R^{2′} independently can be selected from hydrogen, aliphatic, heteroaliphatic, aryl, or heteroaryl; each R³ and R^{3′} independently can be selected from hydrogen, aliphatic, heteroaliphatic, aryl, or heteroaryl; R⁴ can be selected from hydrogen, aliphatic, aryl, heteroaliphatic, or —Si(R)₃, wherein each R is selected from hydrogen, aliphatic, heteroaliphatic, aryl, or heteroaryl; R⁵ can be selected from aryl, heteroaryl, aliphatic, ester, or carboxylic acid; each R⁶, R⁷, R⁸, R⁹, R^{6′}, R^{7′}, R^{8′}, and R^{9′} independently can be selected from hydrogen, aliphatic, heteroaliphatic, aryl, or heteroaryl; or R⁷ or R^{7′} can form a 5-membered or 6-membered ring with R⁸ or R^{8′}, respectively.

In some embodiments, R¹ can be selected from phenyl, pyridinyl, thiophenyl, naphthyl, alkoxyphenyl (e.g., meta-, ortho-, or para-methoxyphenyl), haloalkylphenyl (e.g., meta-, ortho-, or para-trifluoromethylphenyl), halophenyl (e.g., meta-, ortho-, or para-bromophenyl; meta-, ortho-, or para-fluorophenyl; meta-, ortho-, or para-chlorophenyl; or meta-, ortho-, or para-iodophenyl), cyclopropyl, cyclobutyl, cyclopentyl, cyclohexyl, methyl, ethyl, propyl, butyl, pentyl, t-butyl, and the like; each of R², R^{2′}, R³, and R^{3′} independently can be selected from hydrogen, methyl, ethyl, propyl, butyl, pentyl, —OSi(Me)₃, —OSi(Et)₃, —OSi(iPr)₃, and the like; R⁴ can be selected from hydrogen, methyl, ethyl, propyl, butyl, pentyl, phenyl, alkoxy (e.g., methoxy, ethoxy, propoxy, and the like), —Si(Me)₃, —Si(Et)₃, —Si(iPr)₃ and the like; R⁵ can be selected from phenyl, pyridinyl, alkoxyphenyl (e.g., meta-, ortho-, or para-methoxyphenyl), haloalkylphenyl (e.g., meta-, ortho-, or para-trifluoromethylphenyl), halophenyl (e.g., meta-, ortho-, or para-bromophenyl; meta-, ortho-, or para-fluorophenyl; meta-, ortho-, or para-chlorophenyl; or meta-, ortho-, or para-iodophenyl), methyl, ethyl, propyl, butyl, pentyl, —C(O)OH, —C(O)OR, wherein R is selected from hydrogen, aliphatic, heteroaliphatic, aryl, and heteroaryl; and each R⁶, R⁷, R⁸, R⁹, R^{6′}, R^{7′}, R^{8′}, and R^{9′} independently can be selected from hydrogen, alkyl (such as methyl, ethyl, propyl, butyl, pentyl, and the like), alkoxy (such as methoxy, ethoxy, propoxy, and the like), silyloxy (such as —OSi(Me)₃, —OSi(Et)₃, —OSi(iPr)₃, and the like), phenyl, or pyridinyl; or R⁷ or R^{7′} can form a 5-membered ring with R⁸ or R^{8′}, respectively.

In exemplary embodiments, R¹ is selected from methyl, phenyl, pyridinyl, thiophenyl, naphthyl, cyclohexyl, t-butyl, SiMe₃, SiEt₃, 4-methoxyphenyl, 2-methoxyphenyl, 4-bromophenyl, 2-bromophenyl, 4-trifluoromethylphenyl, and 2-trifluoromethylphenyl; each of R² and R³ independently are selected from hydrogen, methyl, —OSi(Me)₃, —OSi(Et)₃, —OSi(iPr)₃; R⁴ is hydrogen; R⁵ is selected from phenyl, 4-methoxyphenyl, 2-methoxyphenyl, 4-bromophenyl, 2-bromophenyl, 4-trifluoromethylphenyl, and 2-trifluoromethylphenyl; R⁶ is selected from hydrogen, R⁷ is hydrogen or forms a 5-membered or 6-membered ring with R⁸; R⁸ is hydrogen, methyl, or forms a 5-membered or 6-membered ring with R⁷; and R⁹ is selected from methyl, methoxy, phenyl, or hydrogen.

In some embodiments, facilitation of a double Diels-Alder reaction between a diynone 200 and a diene 202 using a Lewis acid to generate the bis(cyclohexadienyl) ketone product 500 can be used (Scheme 5). Intermediate 500 contains functionality to initiate a Nazarov cyclization. In some embodiments, a single Lewis acid can, in one-pot, drive the reaction through both the double Diels-Alder and Nazarov cyclizations to generate 6-5-6 tricyclic products 502 and 504. A common obstacle in Nazarov cyclizations is regio-control of the double bond. In some embodiments, an effective strategy for overcoming this difficulty is the incorporation of silicon based groups which can be eliminated to ensure a consistent regiochemical outcome (502, R¹═SiR₃, Scheme 5); however, regiochemical control is not solely limited to compounds comprising silyl groups as other R¹ groups disclosed herein can exhibit regiochemical control.

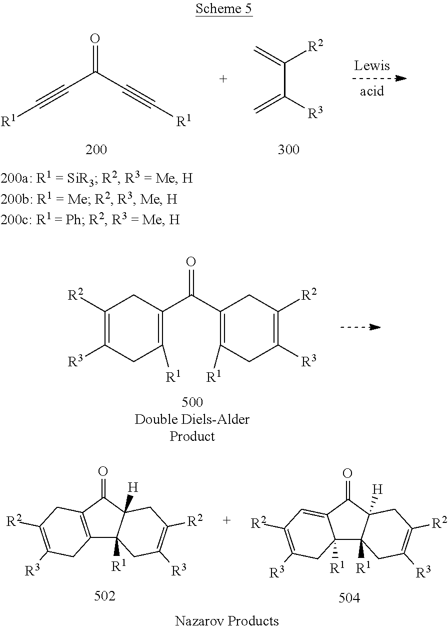

In exemplary embodiments, cyclization conditions with the readily available bis(trimethylsilyl) diynone 200a′ and 2,3-dimethyl-1,3-butadiene 506 were first evaluated while screening a number of common Lewis acids (Table 1). An initial survey using 20 mole % Lewis acid resulted in poor conversion of starting material. In some embodiments, a stoichiometric amount of Lewis acid was used for full conversion of 200a′ to 510 (Table 1).

TABLE 1

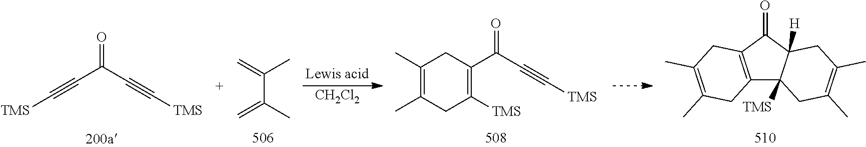

| | | | | |
|---|---|---|---|---|
| Entry | Lewis acid | T (° C.) | Time | Yield (%)^{b} |

| | | | | | |
|---|---|---|---|---|---|
| 1 | ZnCl₂ | 23° C. | 9 | hours | 0 |
| 2 | BF₃•Et₂O | −17° C. | 2 | hours | 0 |
| 3 | SnCl₄ | −78° C. | 75 | minutes | 0 |
| 4 | TiCl₄ | −78° C. | 2 | hours | 0 |
| 5 | FeCl₃ | 0° C. | 1 | hour | 0 |
| 6 | AlCl₃ | 0° C. | 40 | minutes | 30 |
| 7 | EtAlCl₂ | 23° C. | 30 | minutes | 64 |
| 8 | Me₂AlCl | 23° C. | 1.5 | hours | 78 |
| 9 | In(OTf)₃ | 23° C. | 50 | minutes | 0 |
| 10 | BCl₃ | −5 | 10 | min | 58 |

Nazarov reaction conditions: ~0.07 M diynone solution, diene (5 equiv.), Lewis acid (1.2 equiv.), dichloromethane, dry/inert flask, aqueous workup.
^{a}Yield of isoluted Nazarov product.

Reactions disclosed herein can be monitored by TLC and ¹H NMR analysis. In some exemplary embodiments, zinc chloride was not reactive enough to initiate the second Diels-Alder and gave only the monocyclized intermediate 508 (entry 1, Table 1). In some exemplary embodiments, strong Lewis acids decomposed the diene before starting material could be fully converted to 508 (entries 2-5 and 9, Table 1). In some embodiments, aluminum chloride and iron chloride reactions were conducted at 0° C. to increase Lewis acid solubility. Surprisingly, aluminum chloride gave desired product 510 while iron chloride decomposed the diene too quickly. Dimethylaluminum chloride and ethylaluminum chloride gave clean, fast reactions at room temperature although a yield comparison indicated that dimethylaluminum chloride was the most effective in some embodiments (entries 7-8, Table 1). In some embodiments, preliminary screening indicated that mild Lewis Acids were efficacious while stronger Lewis Acids were too harsh for the reaction. A method that employs mild Lewis acids as opposed to more harsh conditions is advantageous as it minimizes side reactions in a more complicated system. In some embodiments, the disclosed methods provide the ability to utilize the synthetic utility of a silicon substituent as silyl substituents (and some others) can be converted into a number of useful functional groups.

A substrate scope for the method also was evaluated in some embodiments. A trimethylsilyl substituted alkyne 200a′ played a role in the regiochemistry of the resulting double bond that forms after the Nazarov cyclization. After the tandem Diels-Alder cyclization occurs generating 600, a Nazarov cyclization results giving 602 (Scheme 6). Without being limited to a particular theory of operation, it is currently believed that the carbocation formed in intermediate 602 upon the Nazarov cyclization is stabilized via the β-silyl effect and is the driving force for the regiochemistry seen in the skipped diene product 510 (Scheme 6).

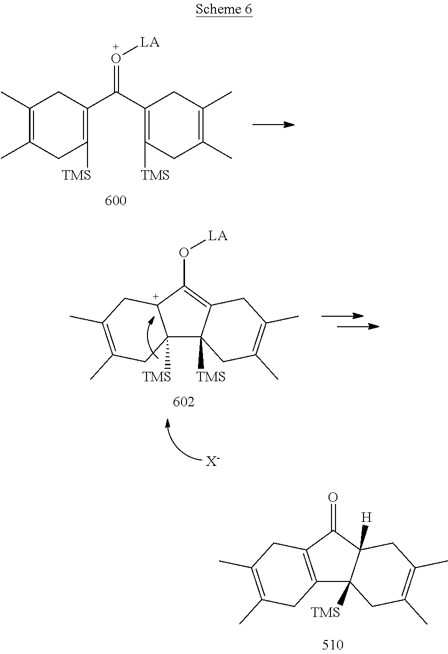

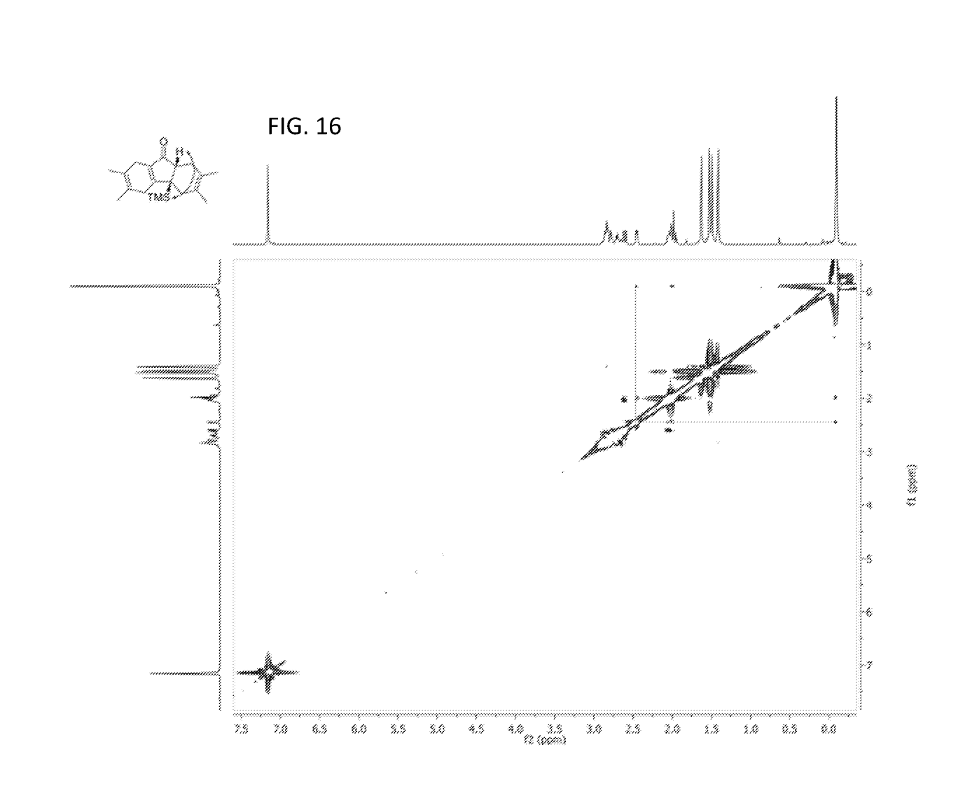

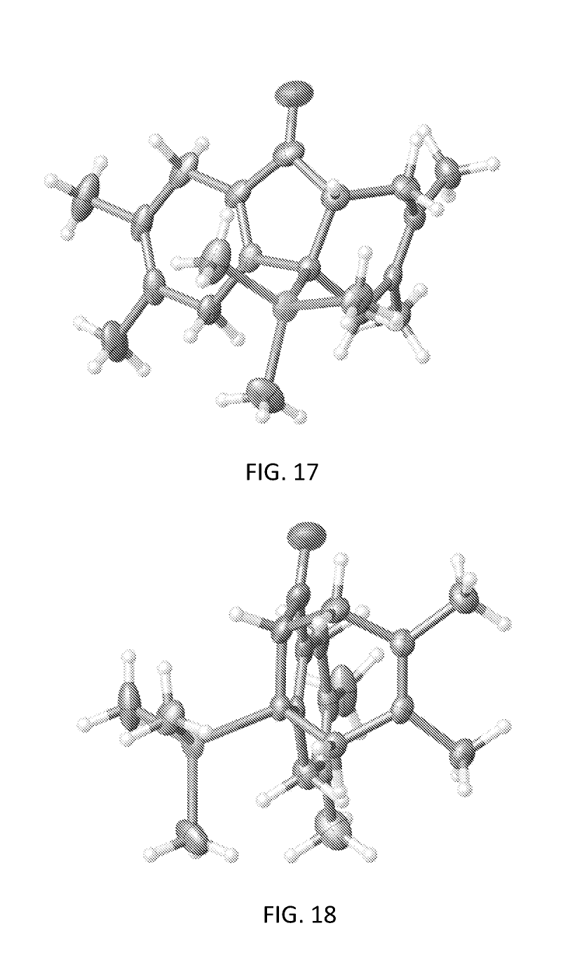

During the course of the method steps described herein, rapid formation of a monocyclized intermediate (similar to that of 508 above, wherein the TMS groups can be present or other R¹ groups can be included). This intermediate can be produced and isolated when the reaction is run at −78° C. then quenched. A second Diels-Alder cyclization then occurs in situ to generate bis-cyclized intermediate 600, which is not isolable in some embodiments. Without being limited to a particular theory, it is currently believed that the intermediate 600 rapidly undergoes the Nazarov cyclization resulting in intermediate 602 (Scheme 6). Stabilization of this carbocationic intermediate via the β-silyl effect can weaken bonds formed between carbon and silicon atoms in silicon-containing intermediates, which results in the regioselective elimination of the silyl moiety, such as the TMS group shown in 602. Upon workup, a product, such as product 510, can be isolated in excellent regio- and diastereoselectivity. The syn relationship between the methine hydrogen (Hₐ) and the TMS substituent in 510 was confirmed by NOESY correlations (see FIG. 16) and single crystal X-ray crystallographic analysis (see FIGS. 17 and 18). Compound 510 has a number of useful functional handles for further synthetic elaboration including two double bonds, a conjugated enone, and the remaining TMS substituent can easily act as a segue towards tertiary alcohols and other functional groups via the Tamao-Fleming oxidation.

A variety of symmetrical diynones can be used to make good yields of the tricyclic products (e.g., entries 1-6, Table 2). Reaction of the symmetrical bis(phenyl)diynone with 2,3-dimethyl-1,3-butadiene resulted in lower yields and formation of the conjugated 1,3-dienone product 504 (entry 4, Table 2). This is not unexpected since upon Nazarov cyclization the resulting carbocation can only undergo elimination via the allylic β-hydrogen. The symmetric dimethyldiynone also performed well to produce 504 in modest yield (entry 3, Table 2). It should be emphasized that not only are five carbon-carbon bonds being generated in this reaction but also three rings, and two vicinal quaternary centers, in a single step reaction (Table 2).

TABLE 2

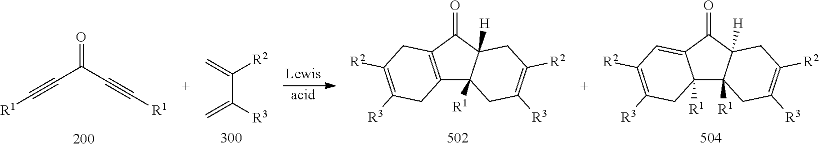

| Entry | Diynone | Lewis acid | Diene | Product | Yield (%)^{a} | Isomeric ratio |
|---|---|---|---|---|---|---|
| 1 | R = TMS 200a′ | Al(CH₃)₂Cl | 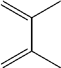 | 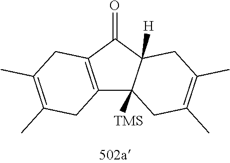 | 78 | 21:1^{b} 19:1^{c} |
| 2 | R = TES 200a″ | Al(CH₃)₂Cl | 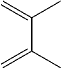 | 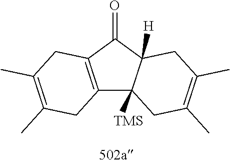 | 46 | 14:1^{b} >20:1^{d} |
| 3 | R = Me 200b | EtAlCl₂ | 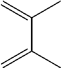 | 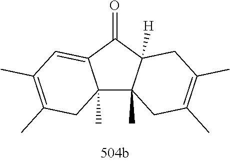 | 35 | 15:1^{b} |
| 4 | R = Ph 200c | EtAlCl₂ | 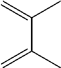 | 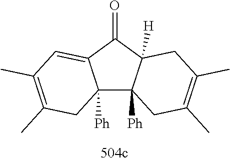 | 27 | >20:1^{d} |
| 5 | R = TMS 200a′ | EtAlCl₂ | 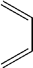 | 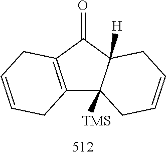 [SMILES: C[Si](C)(C)[C@]12CC=CC[C@H]1C(=O)C1=C2CC=CC1] | 65 | >99:1^{b} |
| 6 | R = TMS 200a′ | Al(CH₃)₂Cl | 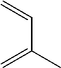 | 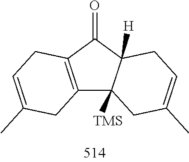 | 54 | 7:1.4:1:1^{b} 7:1:1^{c} |

200a: R¹ = SiR₃; R², R³ = Me, H
200b: R¹ = Me; R², R³ = Me, H
200c: R¹ = Ph; R², R³ = Me, H
^{a}Isolated yield as a mixture of isomers;
^{b}Isomeric ratio of all detectable isomers determined by GC-MS analysis of the crude product;
^{c}Isomeric ratio of all detectable isomers determined by GC-MS analysis after purification;
^{d}Isomeric ratio determined by ¹H NMR.

GC-MS data for crude Nazarov samples was used to determine isomeric ratios (entries 1-6, Table 2). The crude reaction mixtures of 502a′, 502a″, and 512 indicated a high stereoselective preference for the syn diastereomer, minor isomers were not isolated by column chromatography or distillation. GC-MS data for Nazarov product 514 showed four possible isomers, the major product having the expected regiochemistry based on the diene and dienophile energies (entry 6, Table 2). The major isomer and one minor isomer were isolated by column chromatography, the remaining two are suspected of decomposing on the column (entry 6, Table 2). The method worked well for substituted acyclic dienes (entries 1-4 and 6), and even unsubstituted acyclic dienes (entry 5, Table 2).

In the silicon substituted diynone reactions, only the monocyclized intermediate and Nazarov cyclized product were detected with no detection of the double Diels-Alder intermediate 500 (Scheme 5). Without being limited to a particular theory of operation, it is currently believed that the energy barrier of the second Diels-Alder cyclization is higher than both the first Diels-Alder and the Nazarov. However, the alkyl and aryl substituted diynone reactions went through a stable bis-cyclized intermediate 500 which could be isolated in good yield (Scheme 7). Treatment of 500 with ethylaluminum dichloride cleanly provides Nazarov product 504b and 504c.

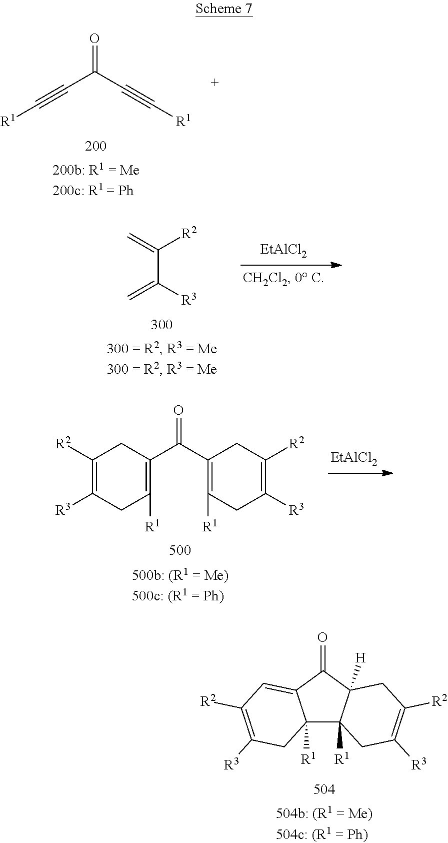

In summary, the first double Diels-Alder/Nazarov tandem cyclization of diynones to yield biologically important 6-5-6 tricyclic scaffolds in a one-pot reaction can been performed using the methods described herein. The disclosed polycyclization methods are highly regioselective and diastereoselective, providing very concise and efficient access to important molecules while also imparting functional handles for further chemical elaboration. In some embodiments, an asymmetric diynone variation will be used, which will incorporate a beta-silyl effect to obtain regioselective control in the Nazarov products.

Exemplary diynones that can be used in certain embodiments are illustrated below.

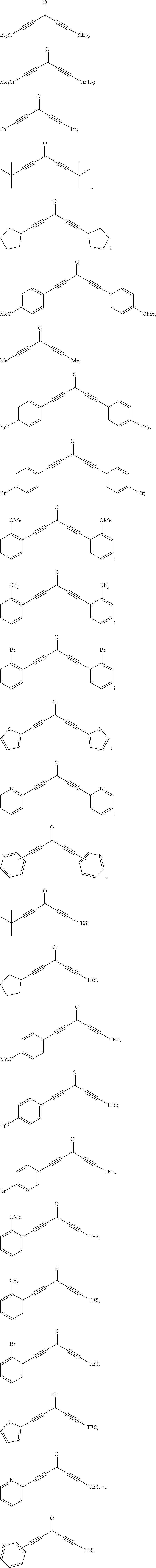

Exemplary dienes that can be used in the methods disclosed herein are illustrated below.

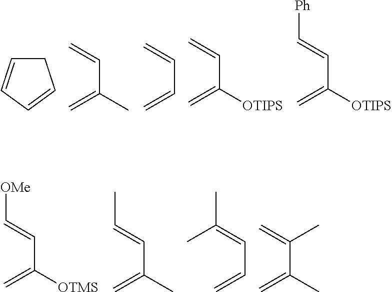

Exemplary multicyclic compounds that can be made using the methods disclosed herein are illustrated below.

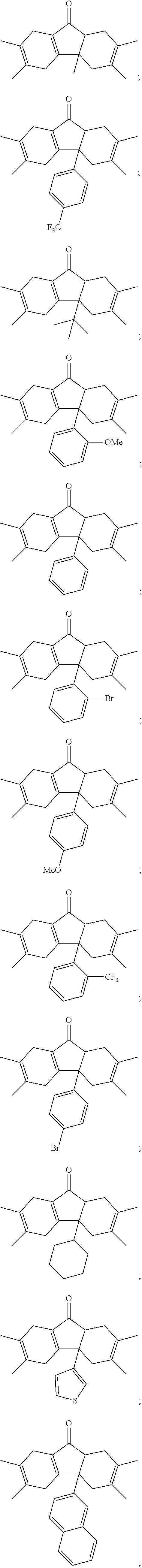

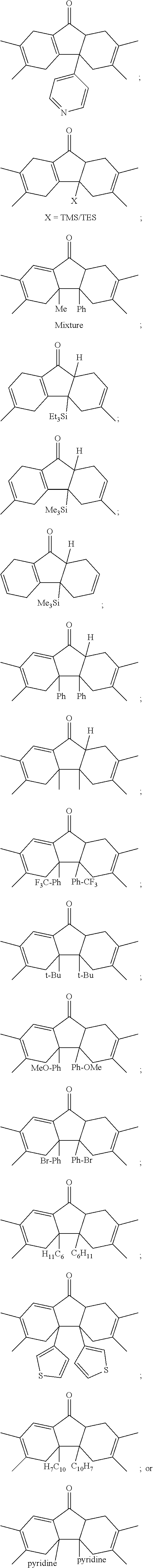

The Diels-Alder of alkynes and dienes has been demonstrated in a number of cases. For example, the cyclization of electron-deficient alkynes, such as 800 with Danishefsky diene 802 results in the formation of aromatic phenol products 806 (Scheme 8). The intermediate generated in this reaction is a 1,4-cyclohexadiene (804), which would be a really useful synthon for future chemistry if it could be isolated. The utilization of a diene that will slow down or prevent aromatization would allow the formation and isolation of the cyclic skipped diene 902 (Scheme 9). It should be noted that 1,4-cyclohexadienes, such as 902 are typically produce under harsh dissolved-metal reduction conditions, being limited in functional group tolerance. Compound 902 provides two useful functionalities, namely, an electron-poor alkene and a neutral alkene, both of which can be chemoselectively transformed into number of useful functional groups. The production of an electron-deficient alkene in the product can undergo another cyclization to generate bicyclic products. The challenge is that tri- and tetrasubstituted alkenes tend to be much less reactive in Diels-Alder cyclization reactions. Without being limited to a particular theory of operation, it is currently believed that the presence of a Lewis-acid catalyst the reaction undergo a double Diels-Alder reaction to generate bicyclic cis-decalin products 904. As illustrated in Schemes 1-4, a number of cis-decalin products can be made using methods described herein.

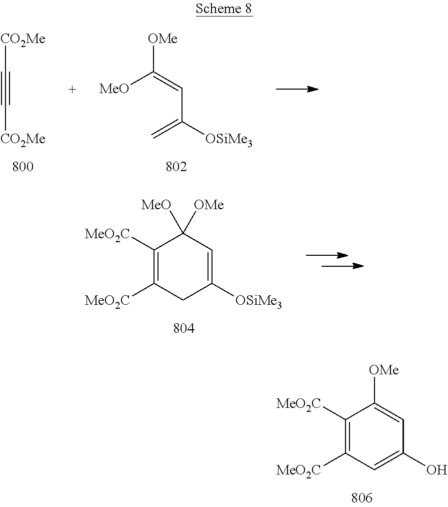

An exemplary scheme is shown below.

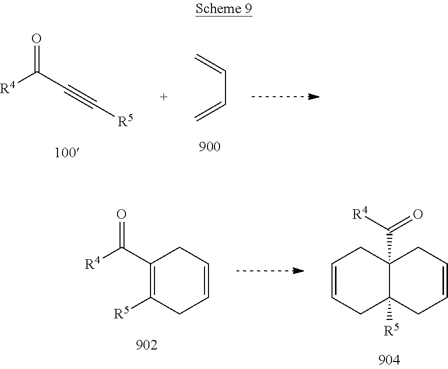

¹H NMR spectrometry can be conducted to evaluate the reaction progress by, for example, mixing one equivalent of α,β-unsaturated alkyne 1000 with excess 2,3-dimethylbutadiene in CDCl₃ in the presence of one equivalent of dimethylaluminum chloride. Such reactions can result in the clean conversion of the alkyne 1000 to monocyclized intermediate 1002 in a very rapid (<10 min) and exothermic fashion (Scheme 10). The second cyclization can be slower, but complete conversion of 1002 to bicyclic product 1004 can be observed after, in some embodiments, 18 hours at room temperature. Another exemplary embodiment is shown in Scheme 11, where two different dienes are used sequentially to provide an unsymmetrical product 1102.

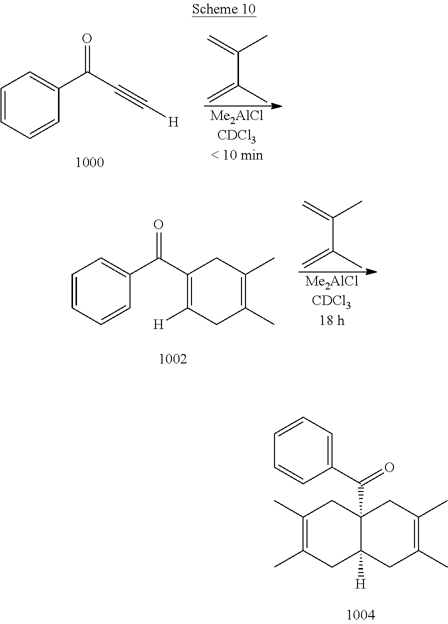

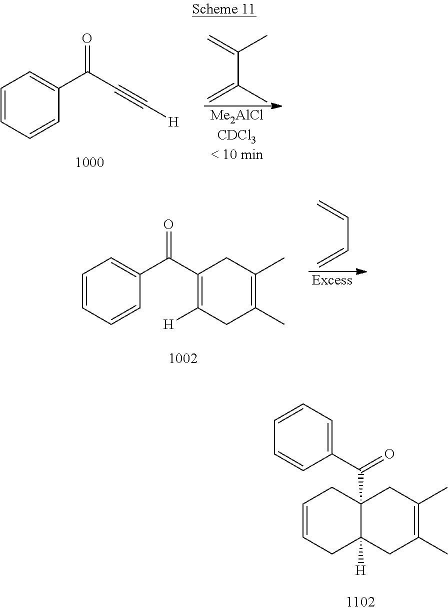

Reaction times were significantly faster with the use of the more reactive ethylaluminum dichloride along with better conversion in many cases. With these conditions and results, the reaction with respect to the alkyne substrate was evaluated. The reaction worked well with a variety of aryl-substituted ynones 100a-g (entries 1-7, Table 3). Alkyl-substituted ynone 100h was also tolerated (entry 8, Table 3). The reaction with more sterically hindered disubstituted alkynes was also explored (Table 3, entries 9-11).

TABLE 3

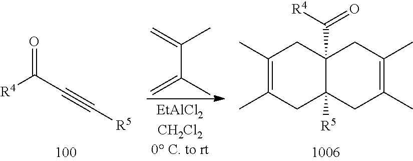

| Entry | 100 | R⁴ | R⁵ | Yield (%) |
|---|---|---|---|---|
| 1 | 100a | Ph | H | 60 |
| 2 | 100b | p-MeO-Ph | H | 18 |
| 3 | 100c | p-Br-Ph | H | 45 |
| 4 | 100d | p-CF₃-Ph | H | |
| 5 | 100e | o-MeO-Ph | H | 26 |
| 6 | 100f | o-Br-Ph | H | 25 |
| 7 | 100g | o-CF₃-Ph | H | |
| 8 | 100h | i-Pr | H | |
| 9 | 100i | Ph | SiMe₃ | |
| 10 | 100j | Ph | Et | |
| 11 | 100h | Ph | Ph | |

A number of dienes also were evaluated and it was determined that many also provided double Diels-Alder cyclization product (Table 4). Cyclopentadiene also worked to provide a mixture of diastereomers. The use of oxygen-functionalized dienes (entry 3, Table 4) provides highly useful functionalize handles for further manipulation.

TABLE 4

| Entry | Diene | Product |
|---|---|---|
| 1 | 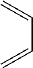 | 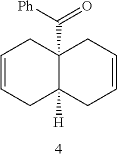 [SMILES: O=C(c1ccccc1)[C@]12CC=CC[C@H]1CC=CC2] |
| 2 | 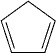 | 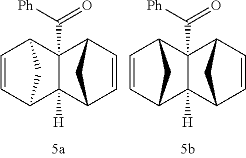 |
| 3 | 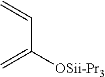 | 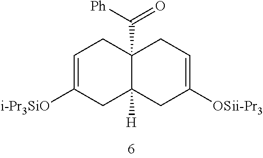 |

Combining two different dienes in a “timed” double Diels-Alder reaction to produce unsymmetrical products can also be performed using the methods disclosed herein. Monocyclized intermediate 1002 was made cleanly with one equivalent of 2,3-dimethylbutadiene (Scheme 12). Once complete conversion of ynone 1000 to 1002 was observed, 5 equivalents of cyclopentadiene was added to the reaction, resulting in the clean formation of unsymmetrical bicyclo product 1200 as a single diastereomer in good yield.

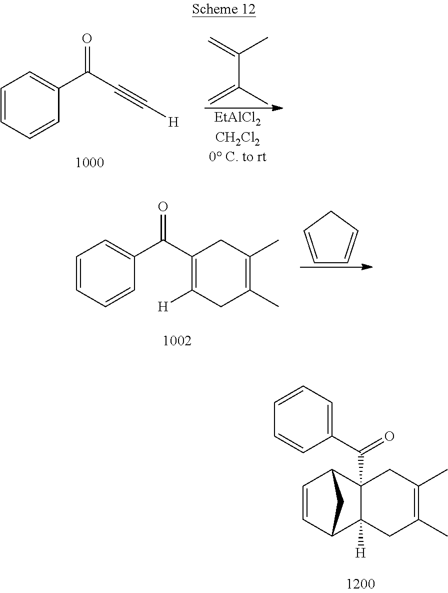

Also disclosed herein are embodiments of an enantioselective variation of this reaction. In particular embodiments, the cyclization of 1000 with 2,3-dimethylbutadiene followed by addition of cyclopentadiene using a BINOL-complexed aluminum catalyst can be used to produce the desired product.

Exemplary alkyne-containing compounds that can be used to make cis-decalin products according to the methods disclosed herein are illustrated below.

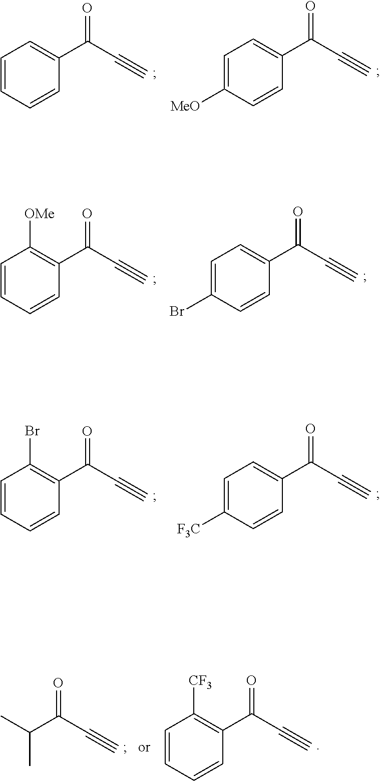

Exemplary cis-decalin products that can be made using the methods disclosed herein can include, but are not limited to, the compounds illustrated below.

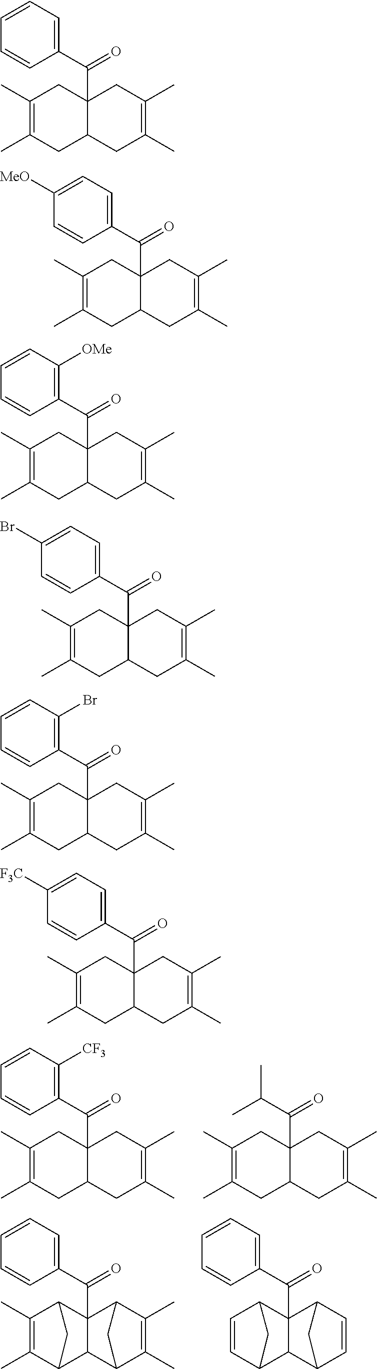

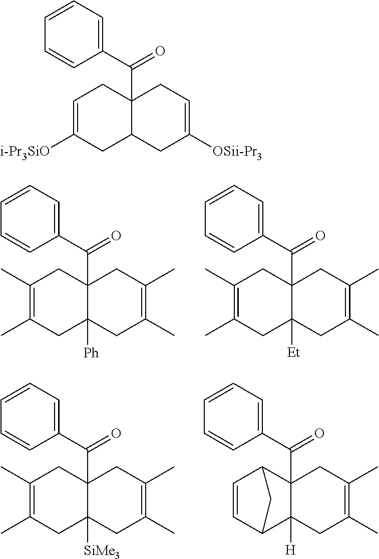

The compounds described herein can be used to make myriad natural and non-natural chemical compounds. In particular disclosed embodiments, the compounds can be used to make terpene products, such as valerane, valeranone, fauronyl acetate, cryptofauronol, kanoknonol, and the like. Such an embodiment is illustrated below in Scheme 13.

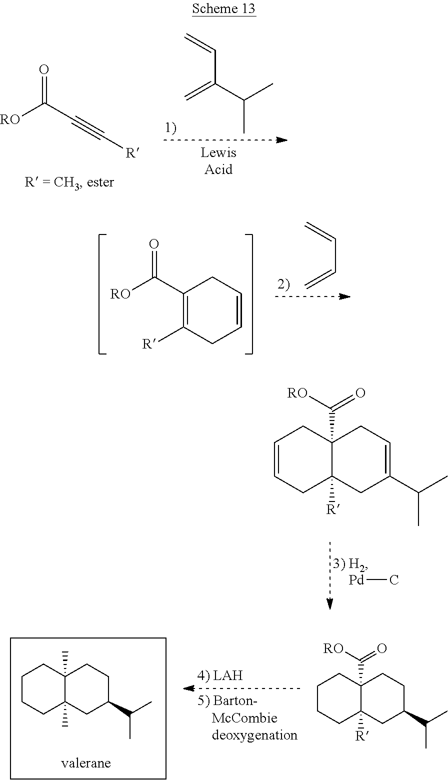

Other exemplary compounds that can be made using the compounds and methods disclosed herein can include, but are not limited to compounds illustrated below.

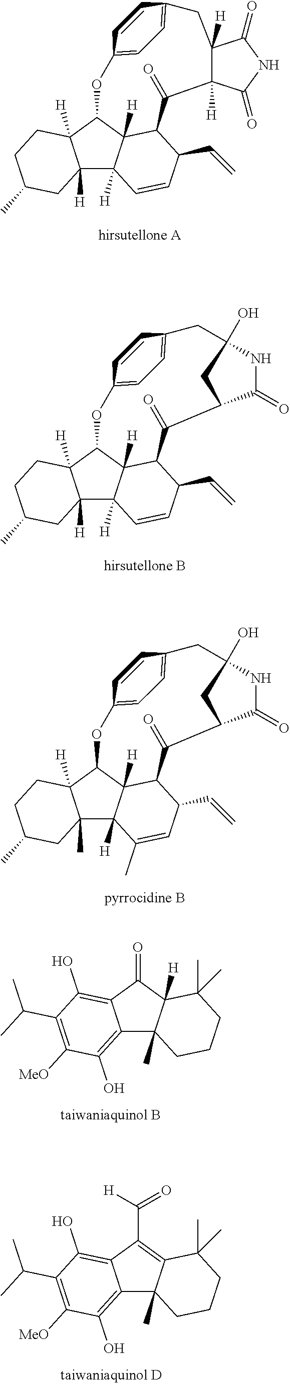

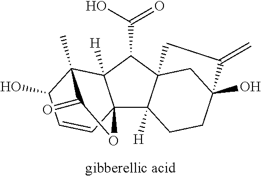

### III. EXAMPLES

General procedures and methods: Reagents were purchased reagent grade from commercial suppliers and used without further purification unless otherwise noted. Dichloromethane and tetrahydrofuran were purified using puresolv MD 5 solvent purification system. Evaporation and concentration in vacuo was done with dry ice—isopropanol using Heidolph G5 rotary evaporator. Where appropriate, reactions were performed in standard, dry glassware under an inert atmosphere of N₂. Column chromatography: Silica gel irregular 60 Å (40-60 micron) from VWR International. Thin-layer chromatography (TLC): glass sheets covered with silica gel 60 F₂₅₄ from Millipore Corporation; visualization by UV light, Anisaldehyde stain or permanganate stain. Mp: Mel-Temp apparatus; uncorrected. JR spectra (cm⁻¹): Thermo Nicolet 6700 FT-JR (diamond ATR), data are reported as cm⁻¹. ¹H and ¹³C NMR: Varian VNMRS 400 MHz, 500 MHz at room temperature in CDCl₃; solvent peaks (7.26 ppm for ¹H and 77.16 ppm for ¹³C, respectively) or C₆D₆; solvent peaks (7.16 ppm for ¹H and 128.06 ppm for ¹³C, respectively) as reference. ESI-TOF MS: Agilent G6230A instrument with purine and HP-0921 as internal calibrants.

Structural Data and Synthetic Procedures:

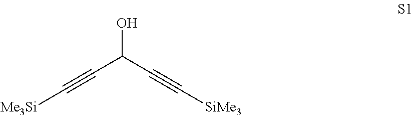 [SMILES: C[Si](C)(C)C#CC(O)C#C[Si](C)(C)C]

S1: Ethynyltrimethylsilane was distilled before use. TLC (EtOAc:Hexanes, 1:10 v/v; R_{f}=0.5; ¹H NMR (500 MHz, CDCl₃) δ 5.11-5.00 (d, J=3.0, 1.5 Hz, 1H), 2.82-2.70 (d, J=4.8 Hz, 1H), 0.17-0.13 (s, J=1.6 Hz, 18H). ¹³C NMR (500 MHz, CDCl₃) δ 102.03, 89.42, 52.89, −0.19. JR (Thermo Nicolet 6700 FT-IR): 3,361 cm⁻¹, 2,960 cm⁻¹, 2,173 cm⁻¹; HRMS (ESI-TOF) (m/z): [M+Na]⁺ calcd for C₁₁H₂₀Si₂O, 249.0950; found, 249.0969.

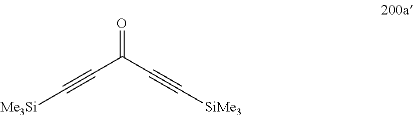 [SMILES: C[Si](C)(C)C#CC(=O)C#C[Si](C)(C)C]

200a′: m.p.: 48-50° C.; TLC (EtOAc:Hexanes, 1:10 v/v; R_{f}=0.8; ¹H NMR (500 MHz, CDCl₃) δ 0.28-0.18 (s, 18H). ¹³C NMR (500 MHz, CDCl₃) δ 160.13, 102.62, 99.24, −0.88. JR (Thermo Nicolet 6700 FT-IR): 2,964 cm⁻¹, 2,154 cm⁻¹, 1,616 cm⁻¹; HRMS (ESI-TOF) (m/z): [M+Na]⁺ calcd for C₁₁H₁₈Si₂ONa, 245.0794; found, 245.0802.

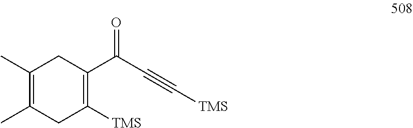 [SMILES: CC1=C(C)CC([Si](C)(C)C)=C(C(=O)C#C[Si](C)(C)C)C1]

Compound 508: To a solution of 200a (93 mg, 0.42 mmol) in CH₂Cl₂ (4 mL) at −78° C. under N₂ was added dimethylaluminum chloride (0.50 mL, 0.50 mmol, 1.0 M in hexanes) followed by 2,3-dimethyl-1,3-butadiene (287 mg, 3.35 mmol). The reaction was stirred until complete by TLC (1-1.5 hours). The reaction was quenched at −78° C. with saturated aqueous NaHCO₃ and extracted with Et₂O. The layers were separated, the organic phase washed with H₂O, brine, and dried over Na₂SO₄. The mixture was filtered through a silica gel plug and the solvent was removed in vacuo to yield crude 508 (112 mg, 88%) as a colorless oil and was not further purified: R_{f}=0.7 (EtOAc:hexanes, 1:10); ¹H NMR (500 MHz, C6D6): δ=3.11 (t, J=8.0 Hz, 2H), 2.78 (t, J=8.1 Hz, 2H), 1.54 (s, 3H), 1.47 (s,3H), 0.31 (s, 9H). 0.05 (s, 9H); ¹³C NMR (125 MHz, C6D6): δ =179.1, 152.8, 142.0, 122.4, 122.4, 103.5, 99.5, 39.9, 35.6, 18.4, 17.9, −0.2, −0.8; JR (film): 2920, 1644 cm-1; HRMS (ESI-TOF) m/z calcd for [C₁₇H₂₈Si₂O⁺H]⁺ 305.1751; found 305.1753.

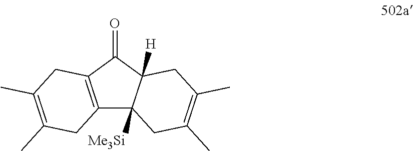

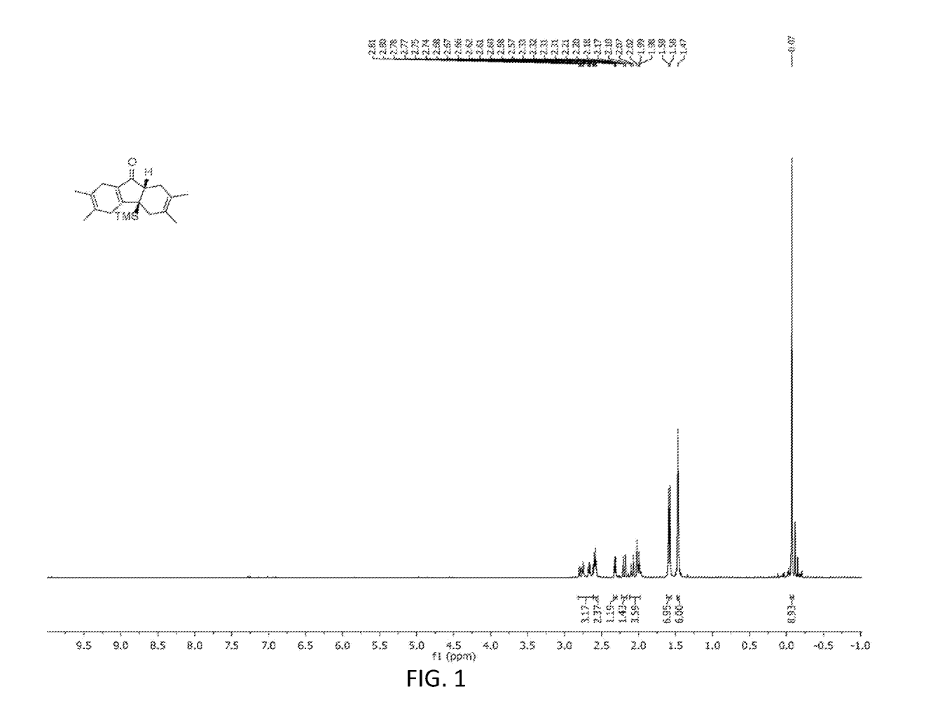

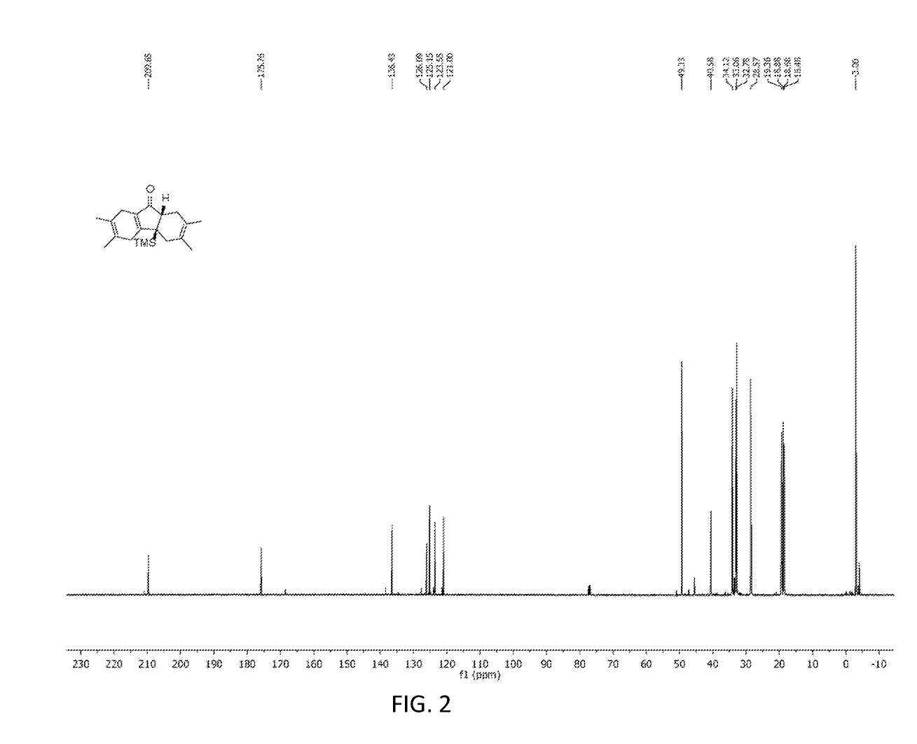

Synthesis of 502a′: To a solution of 200a′ (178 mg, 0.800 mmol) in CH₂Cl₂ (6 mL) under N₂ was added dimethylaluminum chloride (0.96 mL, 0.96 mmol, 1.0 M in hexanes) followed by 2,3-dimethyl-1,3-butadiene (330 mg, 4.0 mmol). The reaction was stirred until complete by TLC (1-1.5 hours). The reaction was quenched at r.t. with saturated aqueous NaHCO₃ and extracted with Et₂O. The layers were separated, the organic phase washed with H₂O, brine, and dried over Na₂SO₄. The mixture was filtered and the solvent was removed in vacuo and the crude product purified by kugelrohr distillation under high vacuum to yield 502a′ (196 mg, 78%) as an off-white solid. The isomeric ratio of the crude product was determined to be 21:1 by GC analysis. The isomeric ratio of the purified product was determined to be 19:1 by GC analysis (GC-MS method: flow=1 mL/min.; inlet=250° C.; 200° C. for 3 minutes, ramp at 2° C./min. to 280° C. and hold for 10 minutes): M.p. 91-95° C.; R_{f}=0.5 (EtOAc:hexanes, 1:10); ¹H NMR (500 MHz, CDCl₃): δ =2.91-2.61 (m, 4H), 2.39 (dd, J=7.1, 3.2 Hz, 1H), 2.26 (dd, J=14.5, 3.3 Hz, 1H), 2.16 (d, J=14.9 Hz, 1H), 2.11-2.00 (m, 2H), 1.66 (s, 2×CH3, 6H), 1.54 (s, 2×CH₃, 6H), 0.00 (s, 9H); ¹³C NMR (100 MHz, CDCl₃): δ =209.6, 175.8, 136.4, 126.1, 125.1, 123.6, 121.0, 49.3, 40.6, 34.1, 33.1, 32.8, 28.6, 19.4, 18.9, 18.7, 18.5, −3.0; IR (film): 2869, 1690, 1631 cm-1; HRMS (ESI-TOF) m/z calcd for [C₂₀H₃₀SiO⁺Na]⁺ 337.1958; found 337.1960. See FIGS. 1 and 2 for NMR spectra.

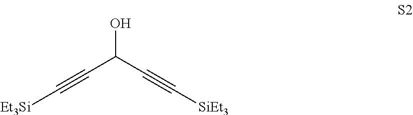

S2: TLC (EtOAc:Hexanes, 1:10 v/v; R_{f}=0.5; ¹H NMR (500 MHz, CDCl₃) δ 5.14-4.96 (s, 1H), 2.75-2.60 (s, 1H), 1.04-0.88 (t, J=8.0 Hz, 18H), 0.65-0.45 (q, J=7.9 Hz, 12H). ¹³C NMR (500 MHz, cdcl₃) δ 103.64, 86.75, 52.87, 7.30, 4.22. JR (Thermo Nicolet 6700 FT-IR): 3,404 cm⁻¹, 2,954 cm⁻¹, 2,872 cm⁻¹, 2,164 cm⁻¹; HRMS (ESI-TOF) (m/z): [M+Na]⁺ calcd for C₁₇H₃₂Si₂O, 331.1889; found, 331.1891.

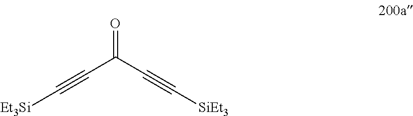

200a″: Dichloromethane was added to a mixture of sieves (1 mass equivalent), celite (1 mass equivalent), and pyridinium chlorochromate (2 equivalents) until mixture was submerged. A dilute solution of S2 in dichloromethane was added slowly to the chromium mixture and stirred overnight or until complete by TLC. The resulting mixture was poured over a celite/silica gel plug. The solvent was removed in vacuo and the crude product purified by column chromatography (ethyl acetate/dichloromethane/hexanes=1:9:90) to yield 200a″ (27%) as a colorless oil: TLC (EtOAc:Hexanes, 1:10 v/v; R_{f}=0.8; ¹H NMR (400 MHz, cdcl₃) δ 1.01-0.92 (dd, J=8.4, 7.5 Hz, 1H), 0.67-0.58 (q, J=8.0 Hz, 1H). ¹³C NMR (500 MHz, cdcl₃) δ 159.67, 104.20, 97.49, 7.13, 3.76. IR (Thermo Nicolet 6700 FT-IR): 2,957 cm⁻¹, 2,872 cm⁻¹, 2,154 cm⁻¹, 1,631 cm⁻¹; HRMS (ESI-TOF) (m/z): [M+Na]⁺ calcd for C₁₇H₃₀Si₂O, 329.1733; found, 329.1728.

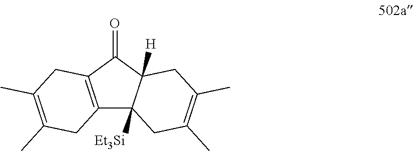

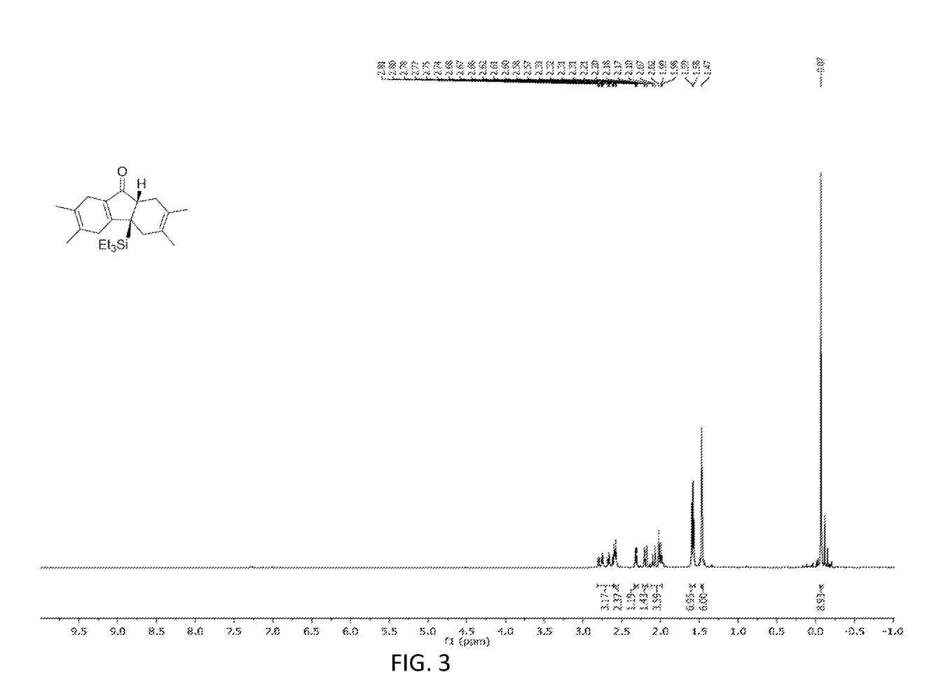

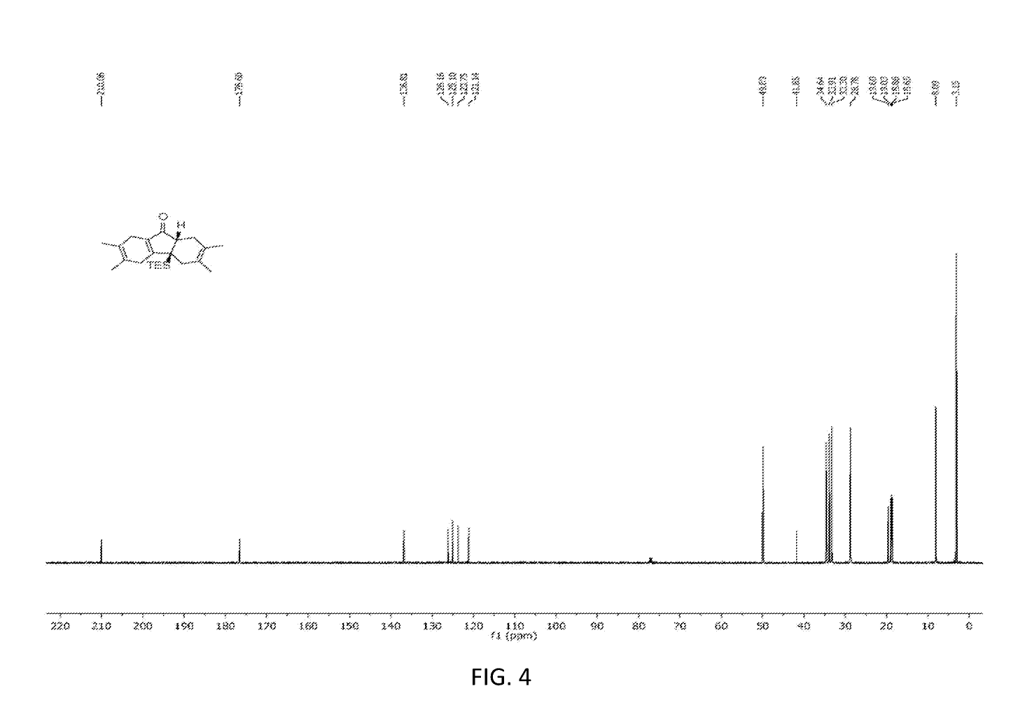

Synthesis of 502a″: To a solution of 200a″ (317 mg, 1.03 mmol) in CH₂Cl₂ (10 mL) under N₂ was added dimethylaluminum chloride (1.24 mL, 1.24 mmol, 1.0 M in hexanes) followed by 2,3-dimethyl-1,3-butadiene (425 mg, 5.20 mmol). The reaction was stirred until complete by TLC (3-3.5 hours). The reaction was quenched at r.t. with saturated aqueous H₂O, brine, and dried over Na₂SO₄. The solvent was removed in vacuo and the crude product purified by column chromatography [pH 7 buffered phosphate silica gel; EtOAc/CH₂Cl₂/hexanes (2:8:90)] to yield 502a″ (171 mg, 46%) as an off-white solid. The isomeric ratio of the crude product was determined to be 14:1 by GC analysis (GC-MS method: flow=1 mL/min.; inlet=250° C.; 200° C. for 3 minutes, ramp at 2° C./min. to 260° C. and hold for 10 minutes). The isomeric ratio of the purified product was determined to be >20:1 by ¹H NMR analysis: M.p. 83-85° C.; R_{f}=0.5 (EtOAc:hexanes, 1:10); ¹H NMR (400 MHz, CDCl3): δ=2.92-2.70 (m, 2H), 2.69-2.61 (m, 2H), 2.57 (dd, J=7.3, 2.7 Hz, 1H), 2.31 (dd, J=14.5, 2.8 Hz, 1H), 2.26-2.10 (m, 3H), 1.70 (s, 6H), 1.57 (s, 6H), 0.96 (t, J=8.0 Hz, 9H), 0.64 (q, J=8.0 Hz, 6H); ¹³C NMR (100 MHz, CDCl3): δ=210.1, 176.6, 136.8, 126.2, 125.1, 123.7, 121.1, 49.9, 41.8, 34.6, 33.9, 33.3, 28.8, 19.6, 19.0, 18.9, 18.7, 8.1, 3.2; IR (film): 2869, 1693 cm-1; HRMS (ESI-TOF) m/z calcd for [C₂₃H₃₆SiO⁺Na]+379.2428; found 379.2439. See FIGS. 3 and 4 for NMR spectra.

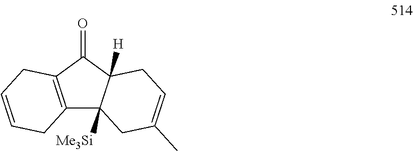 [SMILES: CC1=CC[C@H]2C(=O)C3=C(CC=CC3)[C@@]2([Si](C)(C)C)C1]

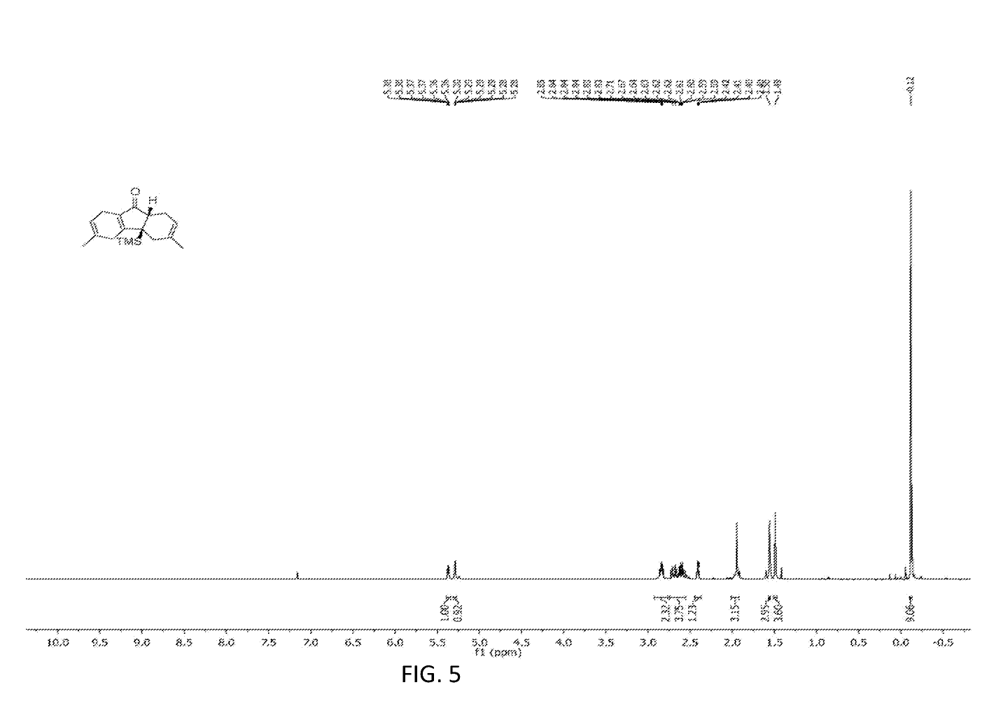

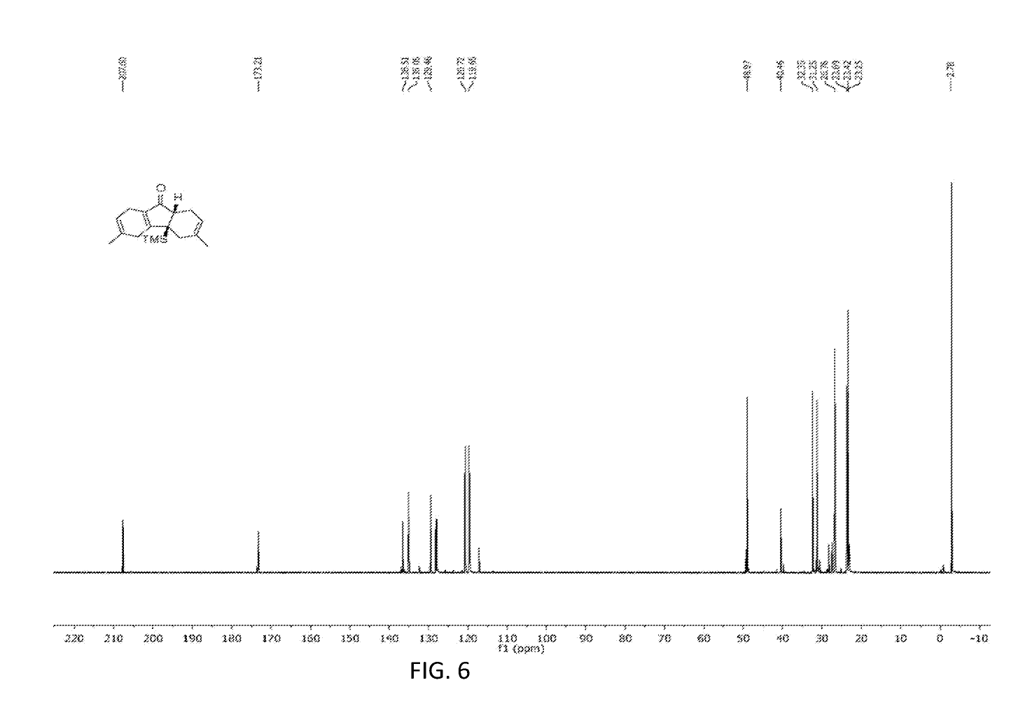

Synthesis of 514: Reaction was conducted in an air free sealed vessel. To a solution of 200a′ (690 mg, 3.10 mmol) in CH₂Cl₂ (30 mL) under N₂ atmosphere in a sealed tube was added dimethylaluminum chloride (4.50 mL, 4.50 mmol, 1.0 M hexanes) followed by excess isoprene (15 mL). The reaction was stirred until complete by TLC (10-12 hours). The reaction was quenched at r.t. with saturated aqueous NaHCO and extracted with Et₂O. The layers were separated, the organic phase washed with H₂O, brine, and dried over Na₂SO₄. The solvent was removed in vacuo and the crude product purified by column chromatography [pH 7 buffered phosphate silica gel, EtOAc/hexanes (4:86)] to yield 514 (476 mg, 54%) as a colorless oil. Four isomers were detected by GC analysis of the crude product in a ratio of 7.1:1.4:1:1 (GC-MS method: flow=1 mL/min; inlet=250° C.; 200° C. for 4 minutes, ramp at 2° C./min. to 260° C. and hold for 10 minutes). The isomeric ratio of the purified product was determined to be 7:1:1 by GC analysis. A small amount of a pure fraction of the major isomer could be isolated by column chromatography for NMR characterization: R_{f}=0.5 (EtOAc:hexanes, 1:10); ¹H NMR (400 MHz, C₆D₆): δ=5.45-5.39 (m, 1H), 5.30-5.24 (m, 1H), 3.02-2.82 (m, 2H), 2.75-2.49 (m, 3H), 2.45 (dd, J=7.5, 2.9 Hz, 1H), 2.03-1.93 (m, 1H), 1.92 (s, 2H), 1.53 (s, 3H), 1.49 (s, 3H), −0.13 (s, 9H); ¹³C NMR (125 MHz, C₆D₆): δ=207.7, 173.0, 136.8, 134.8, 129.1, 120.9, 119.9, 49.0, 40.4, 32.3, 31.2, 26.8, 23.7, 23.5, 23.2, −3.0; IR (film): 2875, 1693 cm⁻¹; HRMS (ESI-TOF) m/z calcd for [C₁₈H₂₆SiO⁺H]⁺ 287.1826; found 287.1821. See FIGS. 5 and 6 for NMR spectra.

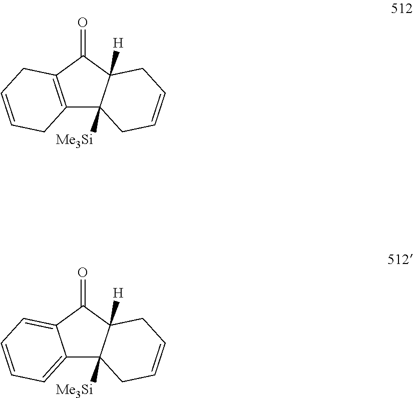 [SMILES: C[Si](C)(C)[C@]12CC=CC[C@H]1C(=O)C1=C2CC=CC1 ; C[Si](C)(C)[C@]12CC=CC[C@H]1C(=O)c1ccccc12]

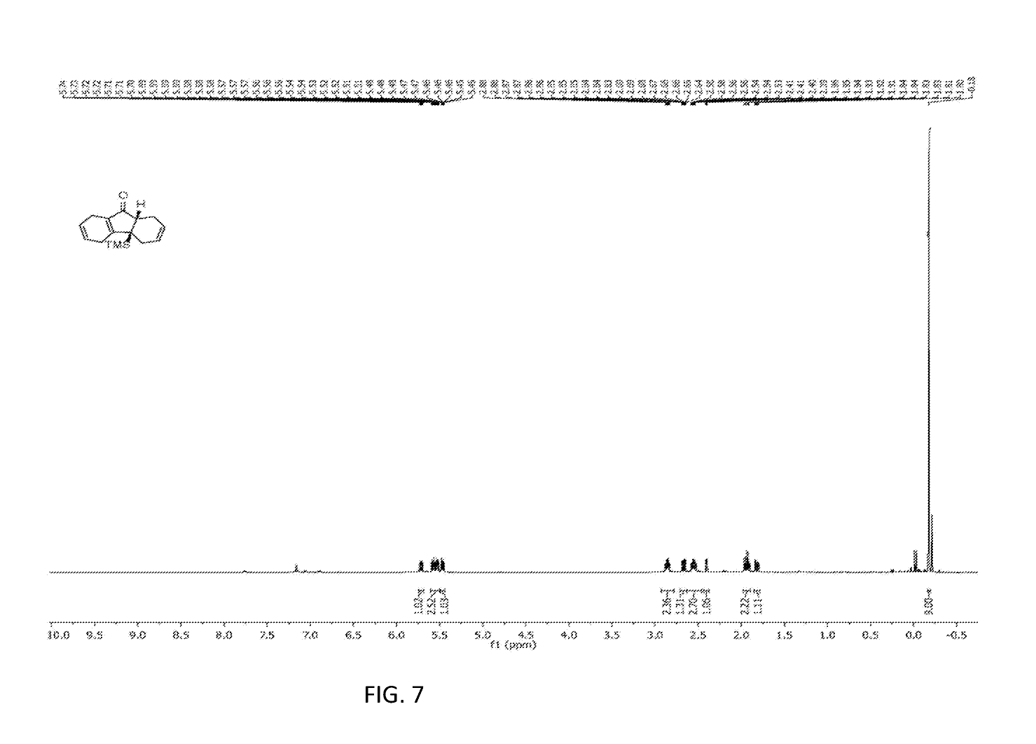

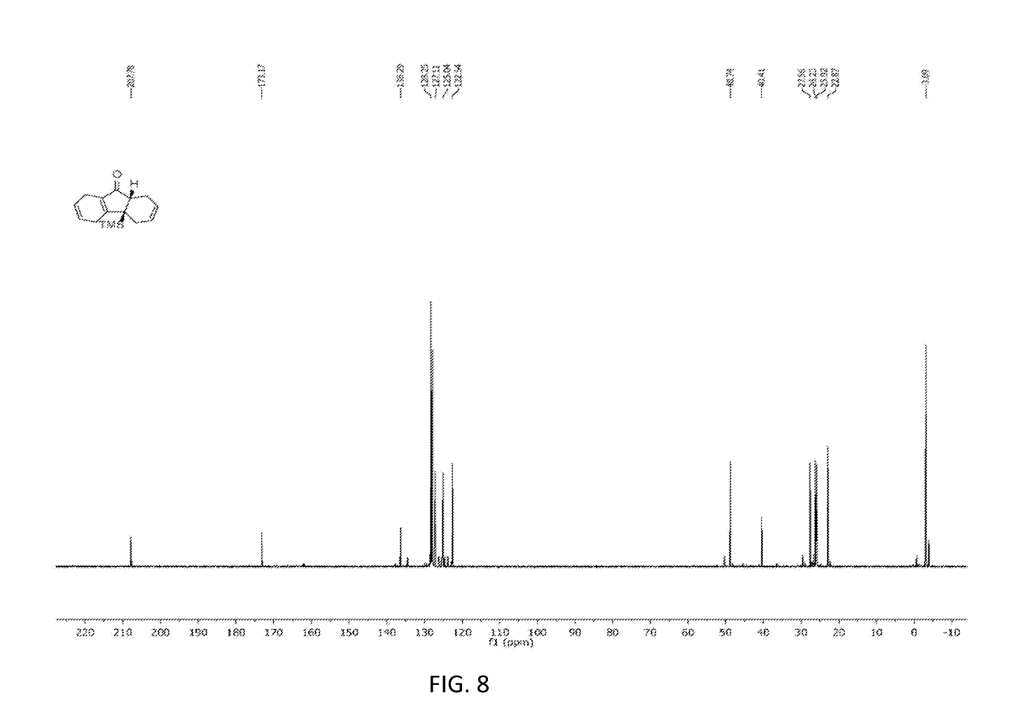

Synthesis of 512: Reaction was conducted in an air free sealed vessel. To a solution of 200a′ (242 mg, 1.09 mmol) in CH₂Cl₂ (6 mL) under N₂ atmosphere in a sealed tube was added ethylaluminum dichloride (1.30 mL, 1.30 mmol, 1.0 M in hexanes). The reaction was cooled to 0° C. and excess 1,3-butadiene was bubbled through the solution for 5 minutes, then the reaction was warmed to r.t. The reaction was stirred until complete by TLC (10-12 hours). The reaction was quenched with saturated aqueous NaHCO₃ and extracted with Et₂O. The layers were separated, the organic phase washed with H₂O, brine, and dried over Na₂SO₄. The solvent was removed in vacuo and the crude product purified by fractional kugelrohr distillation under 0.15 mmHg at 115-125° C. to remove impurities then 135° C. to yield 512 (183 mg, 65%) as a colorless oil. The isomeric ratio of the crude product was determined to be >99:1 by GC analysis (GC-MS method: flow=1 mL/min.; inlet=250° C.; 200° C. for 3 minutes, ramp at 2° C./min. to 260° C. and hold for 10 minutes). Note: After purification by Kugelrohr distillation a ratio of 28:2:1 of 512/oxidized product 512′/unknown isomer was obtained: R_{f}=0.6 (EtOAc:hexanes, 1:10); ¹H NMR (400 MHz, C₆D₆): δ=5.75-5.68 (m, 1H), 5.61-5.44 (m, 3H), 2.94-2.77 (m, 2H), 2.67 (ddd, J=15.0, 6.9, 2.7 Hz, 1H), 2.64-2.45 (m, 2H), 2.40 (dd, J=7.6, 2.6 Hz, 1H), 1.98-1.88 (m, 2H), 1.86-1.78 (m, 1H), −0.18 (s, 9H); ¹³C NMR (125 MHz, C₆D₆): δ=207.8, 173.2, 136.3, 128.3, 127.1, 125.0, 122.5, 48.7, 40.4, 27.6, 26.2, 25.9, 22.9, −3.1; IR (film): 1705, 1246 cm⁻¹; HRMS (ESI-TOF) m/z calcd for [C₁₆H₂₂SiO⁺Na]⁺ 281.1332; found 281.1332. See FIGS. 7 and 8 for NMR spectra of compound 512.

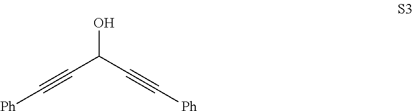 [SMILES: OC(C#Cc1ccccc1)C#Cc1ccccc1]

S3: Phenylacetylene was distilled before use. m.p.: 62-66° C. (lit. 69-70° C.); TLC (EtOAc:Hexanes, 1:10 v/v; R_{f}=0.4; ¹H NMR (500 MHz, cdcl₃) δ 7.60-7.48 (m, 4H), 7.42-7.27 (m, 6H), 5.84-5.53 (s, 1H), 3.36-3.03 (s, 1H). ¹³C NMR (500 MHz, cdcl₃) δ 131.91, 128.84, 128.31, 121.98, 86.16, 84.62, 53.18., IR (Thermo Nicolet 6700 FT-1R): 3,251 cm⁻¹, 2,209 cm⁻¹, 1,484 cm⁻¹; HRMS (ESI-TOF) (m/z): [M+Na]⁺ calcd for C₁₇H₁₂O, 255.0786; found, 255.0782.

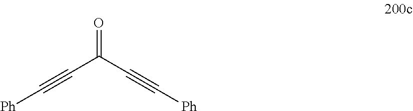 [SMILES: O=C(C#Cc1ccccc1)C#Cc1ccccc1]

200c: m.p.: 59-62° C. (lit. 63.4-64.2° C.); TLC (EtOAc:Hexanes, 1:10 v/v; R_{f}=0.4; ¹H NMR (500 MHz, cdcl₃) δ 7.69-7.63 (d, 2H), 7.53-7.47 (t,1H), 7.45-7.38 (d, J=8.4, 6.9 Hz, 2H). ¹³C NMR (500 MHz, cdcl₃) δ 160.90, 133.47, 131.33, 128.82, 119.49, 91.75, 89.54. IR (Thermo Nicolet 6700 FT-IR): 2,209 cm⁻¹, 2,163 cm⁻¹, 1,603 cm⁻¹, 1,582 cm⁻¹; HRMS (ESI-TOF) (m/z): [M+Na]⁺ calcd for C₁₇H₁₀O, 253.0629; found, 253.0622.

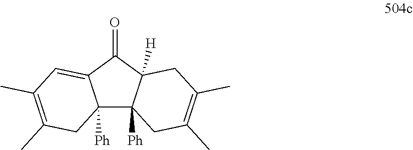

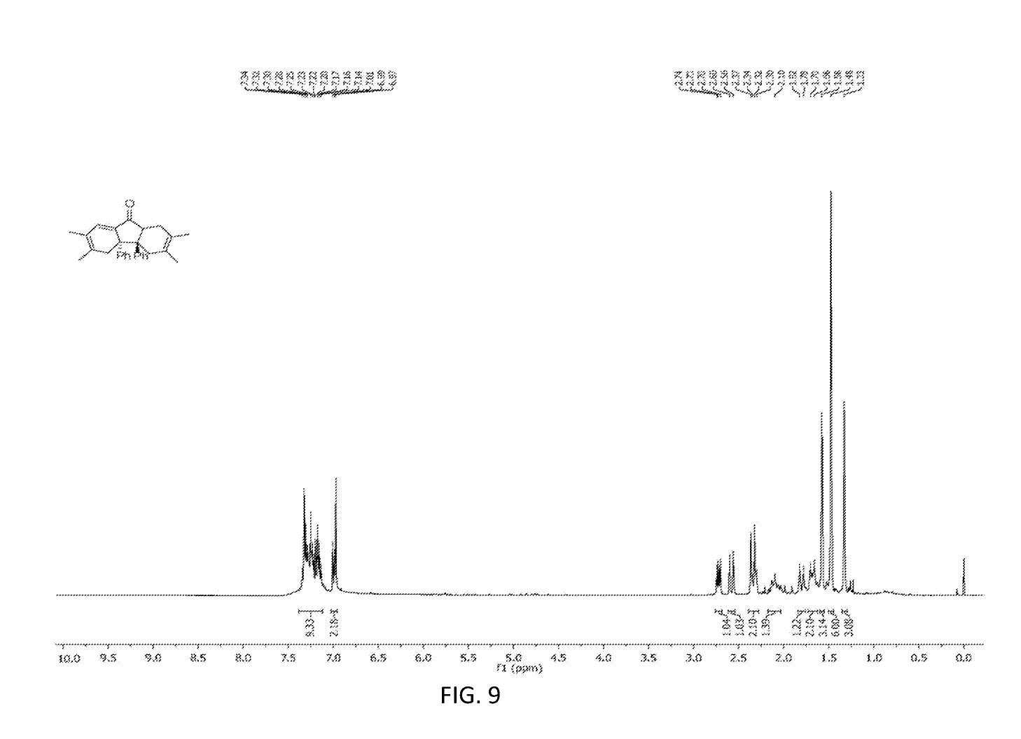

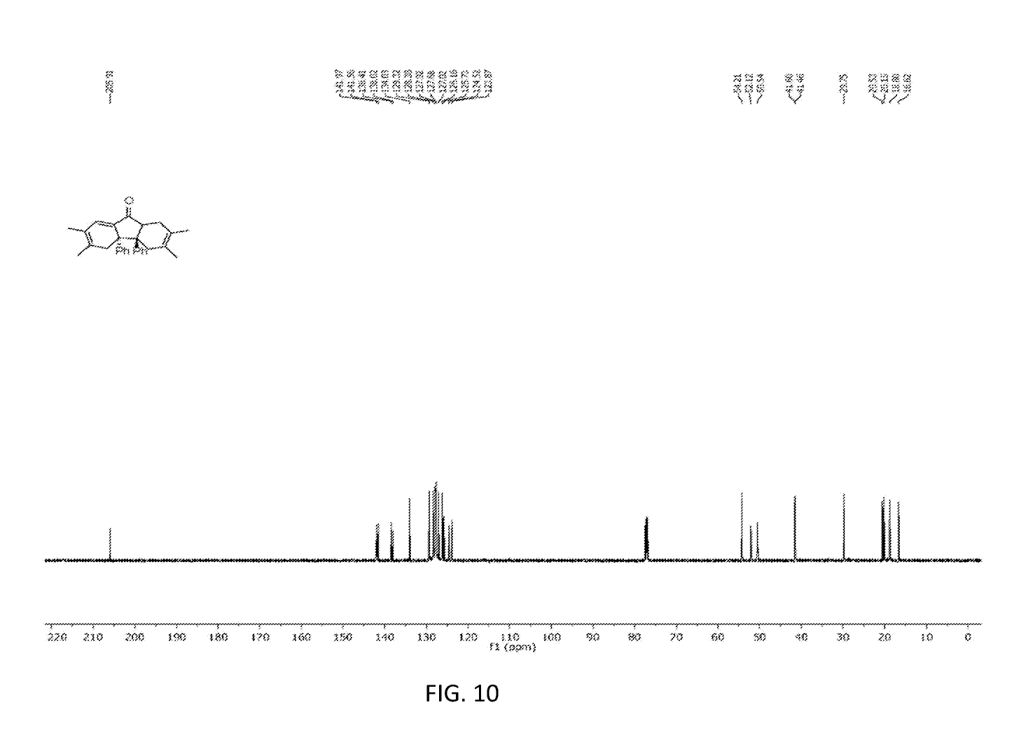

Synthesis of 504c: To a solution of 200c (197 mg, 0.856 mmol) in CH₂Cl₂ (6 mL) under N₂ atmosphere was added ethylaluminum dichloride (1.0 mL, 1.0 mmol, 1.0 M in hexanes) followed by 2,3-dimethyl-1,3-butadiene (351 mg, 4.30 mmol). The reaction was stirred until complete by TLC (24 hours). The reaction was quenched at r.t. with saturated aqueous NaHCO₃ and extracted with Et₂O. The layers were separated, the organic phase washed with H₂O, brine, and dried over Na₂SO₄. The solvent was removed in vacuo and the crude product purified by column chromatography [pH 7 buffered phosphate silica gel, CH₂Cl₂/EtOAc/hexanes (30:1:69)] to yield 504c (92 mg, 27%) as an orange solid. The isomeric ratio of the crude product was determined to be >20:1 by GC analysis (GC-MS method: flow=1 mL/min.; inlet=250° C.; 220° C. for 3 minutes, ramp at 1° C./min. to 280° C. and hold for 10 minutes): R_{f}=0.6 (EtOAc:hexanes, 1:10); ¹H NMR (400 MHz, CDCl3): δ=7.39-7.08 (m, 9H), 7.03-6.96 (m, 1H), 6.98 (s, 1H), 2.73 (dd, J=10.8, 5.7 Hz, 1H), 2.59 (d, J=16.4 Hz, 1H), 2.39-2.29 (m, 2H), 2.16-2.04 (m, 1H), 1.81 (d, J=16.6 Hz, 1H), 1.74-1.65 (m, 1H), 1.58 (s, 3H), 1.48 (s, 2×CH3, 6H), 1.34 (s, 3H); ¹³C NMR (100 MHz, CDCl3): δ=205.9, 142.0, 141.6, 138.4, 138.0, 134.0, 129.3, 128.4, 127.9, 127.7, 127.0, 126.2, 125.7, 124.5, 123.9, 54.2, 52.1, 50.5, 41.6, 41.5, 29.8, 20.5, 20.2, 18.8, 16.6; IR (film): 2900, 1696, 1564, 1439 cm-1; HRMS (ESI-TOF) m/z calcd for [C₂₉H₃₀O⁺Na]⁺ 417.2189; found 417.2182. See FIGS. 9 and 10 for NMR spectra.

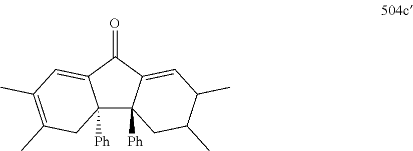

504c′: To a solution of 200c (215 mg, 0.934 mmol) in CH₂Cl₂ (10 mL) under N2 atmosphere at −15° C. was added boron trichloride (0.935 mL, 0.935 mmol, 1 M in hexanes) followed by 2,3-dimethyl-1,3-butadiene (385 mg, 4.67 mmol). After 3 h the reaction was warmed to 0° C. and stirred until complete by TLC (an additional 4.5 hours). The reaction was quenched at r.t. with saturated aqueous NH₄Cl and extracted with Et₂O. The layers were separated, the organic phase washed with H₂O, brine, and dried over Na₂SO₄. The solvent was removed in vacuo and the crude product purified by column chromatography [pH 7 buffered phosphate silica gel, Et₂O /hexanes (1:12)] to yield a single isomer of 504c′ (41 mg, 11%) as a yellow solid: R_{f}=0.33 (EtOAc:hexanes, 1:20); ¹H NMR (500 MHz, C6D6): δ=7.34 (d, J=7.8 Hz, 2H), 7.26 (d, J=7.2 Hz, 2H), 7.20-7.07 (m, 4H), 7.06 (s, 1H), 7.05-6.97 (m, 2H), 6.95 (d, J=3.2 Hz, 1H), 2.45 (d, J=16.5 Hz, 1H), 2.07-1.97 (m, 2H), 1.43-1.35 (m 1H), 1.28 (s, 1H), 1.21 (s, 1H), 1.09-1.01 (m, 1H), 0.70 (t, J=12.6 Hz, 1H), 0.62 (d, J=7.1 Hz, 3H), 0.56 (d, J=6.6 Hz, 3H); ¹³C NMR (125 MHz, C6D6): δ=192.9, 147.4, 145.9, 143.7, 140.7, 140.4, 137.9, 134.4, 128.4, 128.2, 128.0, 127.6, 127.1, 126.4, 125.3, 54.5, 52.5, 42.9, 42.3, 39.5, 34.3, 20.3, 20.0, 17.6, 16.2; IR (film): 2944, 1688; 1641, 1567 cm-1; HRMS (ESI-TOF) m/z calcd for [C29H30O+H]+ 395.2369; found 395.2373.

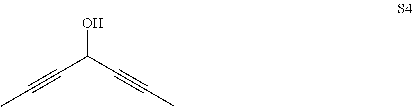

S4: m.p.: 103-105° C. (lit. 152-153° C.); TLC (EtOAc:Hexanes, 1:10 v/v; R_{f}=0.4; ¹H NMR (500 MHz, cdcl₃) δ 5.08-5.02 (dt, J=6.9, 2.3 Hz, 1H), 2.15-2.07 (m, 1H), 1.88-1.86 (d, J=2.3 Hz, 9H). ¹³C NMR (500 MHz, cdcl₃) δ 80.50, 77.14, 52.13, 3.46. IR (Thermo Nicolet 6700 FT-1R): 3,190 cm⁻¹, 2,291 cm⁻¹, 2,224 cm⁻¹; HRMS (ESI-TOF) (m/z): [M+Na]⁺ calcd for C₇H₈O, X; found, X.

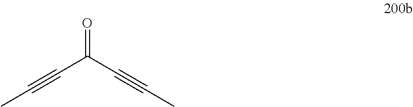

200b: m.p.: 78-80° C. (lit. 79.7-80° C.); TLC EtOAc:Hexanes, 1:10 v/v; R_{f}=0.4; ¹H NMR (400 MHz, cdcl₃) δ 2.10-1.72 (s, 1H). ¹³C NMR (101 MHz, cdcl₃) δ 161.03, 90.52, 81.26, 4.03. IR (Thermo Nicolet 6700 FT-IR): 2,209 cm⁻¹, 1,619 cm⁻¹; HRMS (ESI-TOF) (m/z): [M+Na]⁺ calcd for C₇H₆O, 129.0316; found, 129.0290.

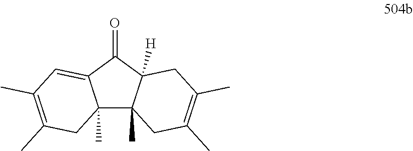

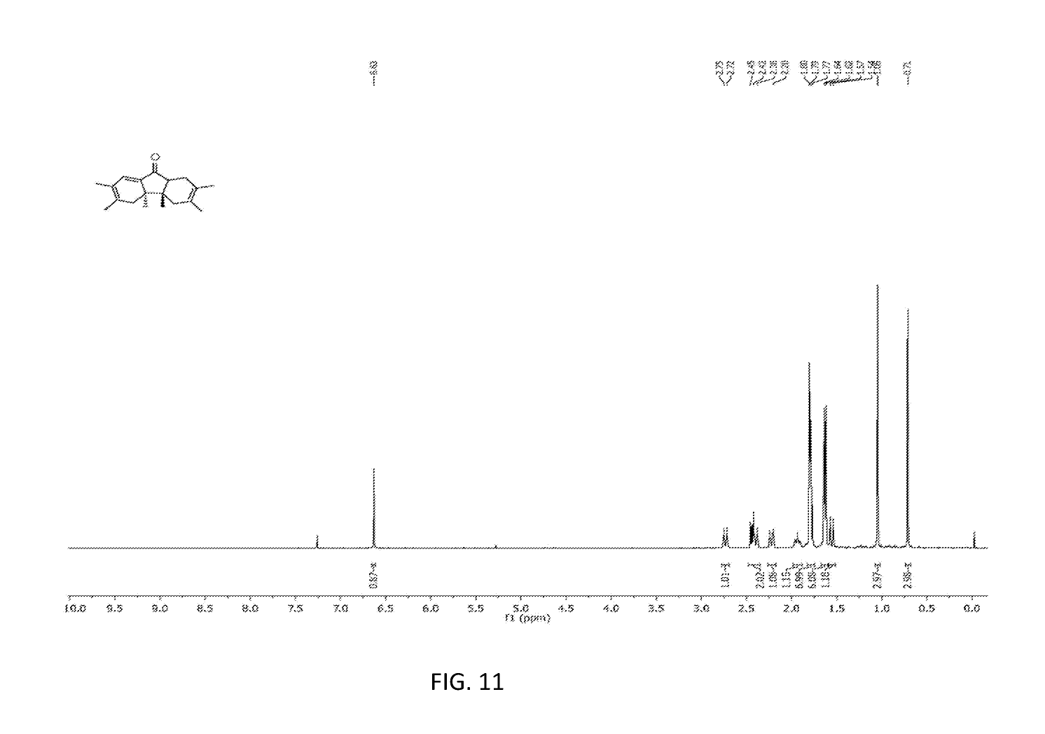

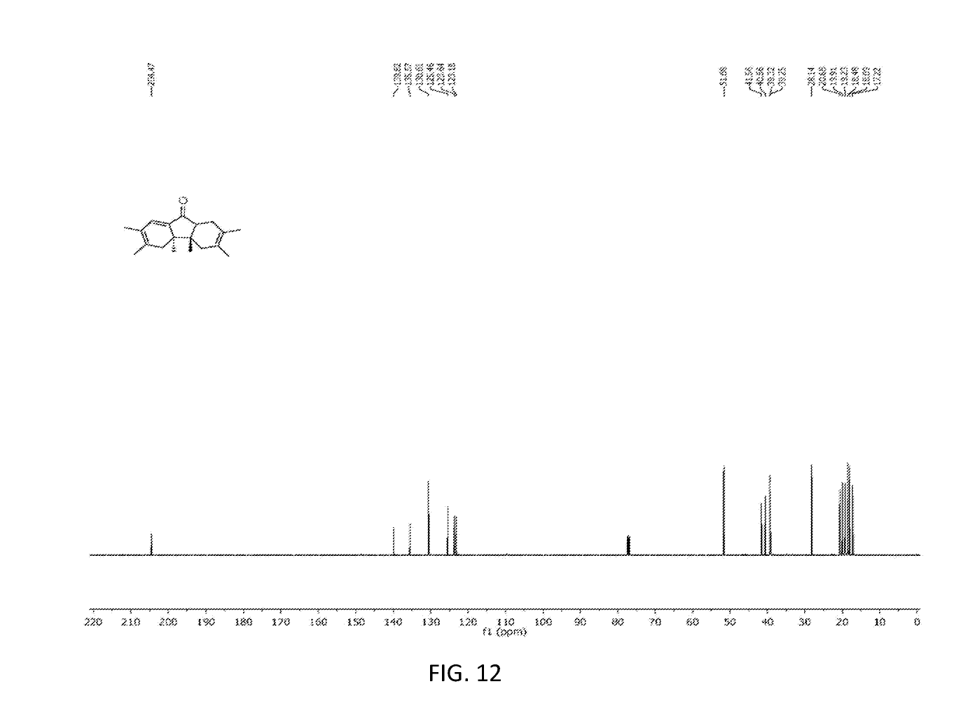

Synthesis of 504b: To a solution of 200b (173 mg, 1.63 mmol) in CH₂Cl₂ (6 mL) under N₂ atmosphere was added ethylaluminum dichloride (1.92 mL, 1.92 mmol, 1 M in hexanes) followed by 2,3-dimethyl-1,3-butadiene (670 mg, 8.20 mmol). The reaction was stirred until complete by TLC (13-14 hours). The reaction was quenched at r.t. with saturated aqueous NaHCO₃ and extracted with Et₂O. The layers were separated, the organic phase washed with H₂O, brine, and dried over Na₂SO₄. The solvent was removed in vacuo and the crude product purified by column chromatography [pH 7 buffered phosphate silica gel, CH₂Cl₂/EtOAc/hexanes (16:1:83)] to yield 504b (153 mg, 35%) as an off-white solid. The isomeric ratio of the crude product was determined to be 15:1 by GC analysis (GC-MS method: flow=1 mL/min.; inlet=250° C.; 200° C. for 3 minutes, ramp at 2° C./min. to 260° C. and hold for 10 minutes): M.p. 102-105° C.; R_{f}=0.5 (EtOAc:hexanes, 1:10); 1H NMR (500 MHz, CDCl3): δ=6.63 (s, 1H), 2.73 (d, J=17.2 Hz, 1H), 2.44 (dd, J=11.2, 5.7 Hz, 1H), 2.40 (d, J=17.1 Hz, 1H), 2.22 (dd, J=17.4, 5.5 Hz, 1H), 1.98-1.88 (m, 1H), 1.80 (s, 3H), 1.79 (s, 3H), 1.78 (d, J=16.4 Hz, 1H), 1.64 (s, 3H), 1.62 (s, 3H), 1.55 (d, J=16.4 Hz, 1H), 1.05 (s, 3H), 0.71 (s, 3H); ¹³C NMR (100 MHz, CDCl3): δ=204.5, 139.8, 135.6, 130.6, 125.5, 123.6, 123.2, 51.7, 41.6, 40.6, 39.3, 39.2, 28.1, 20.7, 19.9, 19.2, 18.5, 18.1, 17.2; IR (film): 2903, 1702, 1579 cm-1; HRMS (ESI-TOF) m/z calcd for [C19H26O+H]+ 271.2056; found 271.2055. Using an asymmetric diynone containing a TMS substituent, the regiochemistry of the resulting double bond that is formed from the Nazarov cyclization step can be controlled, as illustrated below. See FIGS. 11 and 12 for NMR spectra.

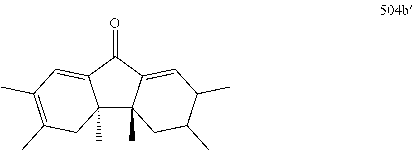

504b′: To a solution of 200c (142 mg, 1.33 mmol) in CH₂Cl₂ (12 mL) at −15° C. under N₂ atmosphere was added boron trichloride (1.33 mL, 1.33 mmol, 1 M in hexanes) followed by 2,3-dimethyl-1,3-butadiene (552 mg, 6.70 mmol). The reaction was stirred until complete by TLC (3 hours). The reaction was quenched at −15° C. with saturated aqueous NH₄Cl and extracted with Et₂O. The layers were separated, the organic phase washed with H₂O, brine, and dried over Na₂SO₄. The solvent was removed in vacuo and the crude product purified by column chromatography [pH 7 buffered phosphate silica gel, H2Cl2/EtOAc/hexanes (18:2:80)] to yield 504b′ (115 mg, 32%) as a yellow oil. Four isomers were detected by GC analysis of the crude product in a ratio of 71:12:5:1 (GC-MS method: flow=1 mL/min.; inlet=250° C.; 200° C. for 3 minutes, ramp at 2° C./min. to 260° C. and hold for 10 minutes). The isomeric ratio of the purified product was determined to be 22:1 by GC analysis: R_{f}=0.34 (EtOAc:hexanes, 1:20); 1H NMR (400 MHz, CDCl3): δ=6.65 (s, 1H), 6.43 (d, J=2.9 Hz, 1H), 2.73 (d, J=17.1 Hz, 1H), 1.93-1.82 (m, 1H), 1.81 (d, J=17.5 Hz, 1H), 1.77 (s, 2×CH₃, 6H), 1.59-1.51 (m, 1H), 1.48 (dd, J=12.3, 12.1 Hz, 1H), 1.36 (dd, J=12.3, 3.1 Hz, 1H), 1.08 (d, J=7.2 Hz, 3H), 1.02 (s, 3H), 1.00 (d, J=6.4 Hz, 3H), 0.88 (s, 3H); ¹³C NMR (100 MHz, CDCl3): δ=193.8, 146.7, 141.1, 137.2, 135.5, 131.2, 123.2, 43.3, 42.5, 39.0, 38.23, 38.22, 33.1, 27.3, 21.6, 20.7, 20.6, 18.7, 17.4; IR (film): 2956, 1692; 1649, 1583 cm-1; HRMS (APCI-TOF) m/z calcd for [C₁₉H₂₆O⁺H]⁺ 271.2056; found 271.2060.

In some embodiments, different dienes can be used in a sequential fashion to produce asymmetric reaction products. In some embodiments, the regiochemistry of the reaction can be controlled based on what order the different dienes are added to the reaction mixture.

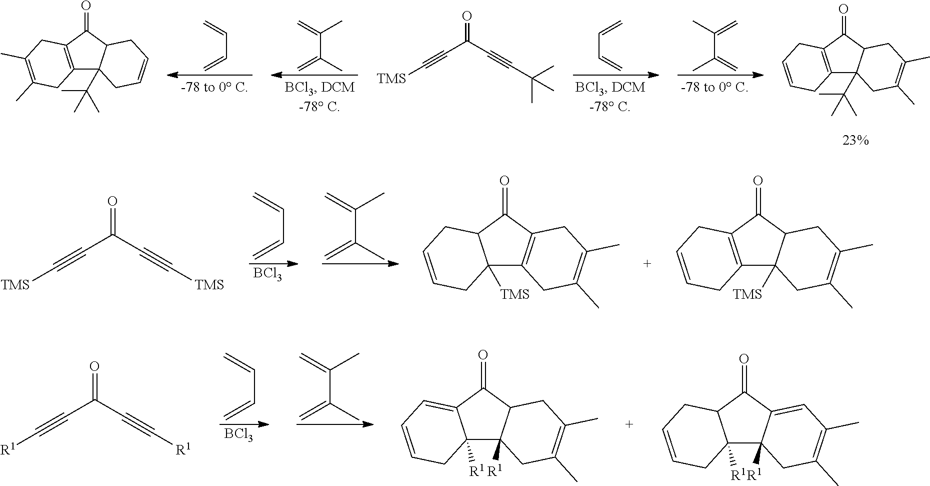

In some embodiments, asymmetric diynones that contain different substituents can be used, such as is illustrated below. Other groups corresponding to R¹ can be used for the diynones.

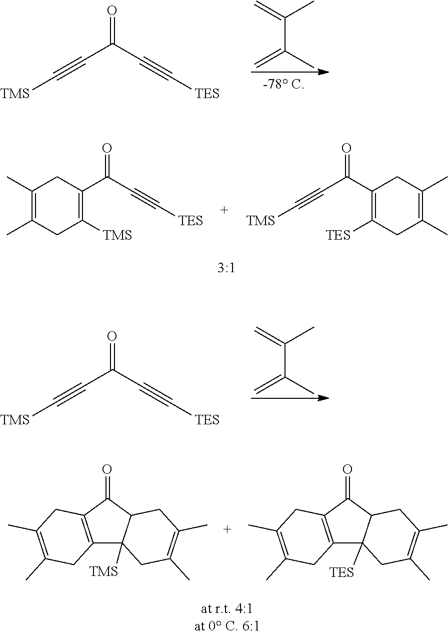

General Procedures and Methods for Tandem Diels-Alder Reactions: Reagents were purchased reagent grade from commercial suppliers and without further purification. Evacuation and concentration in vacuo was done with dry ice/isopropanol on a Heidolph G5 rotary evaporator. All reactions were carried out under an inert atmosphere of N₂ in flame-dried glassware with magnetic stirrers. Thin-layer chromatography (TLC): glass-backed silica gel 60 F₂₅₄ from Millipore Corporation; visualization by UV light and anisaldehyde stain. Distillation: Kugelrohr GKR-51 from Büchi. ¹H and ¹³C NMR: Varian VNMRS 400, 500, or Varian MR-400 at room temperature in CDCl₃; solvent peaks (7.26 ppm for ¹H and 77.0 ppm for ¹³C, respectively) as reference.

General Procedure for Tandem Diels-Alder Reactions: To a solution of ynones 100a-100g (1.0 equiv.) and 2,3-dimethyl-1,3-butadiene (5.0 equiv.) in DCE (10 mL) at 0° C. was added AlEtCl₂ (1.0 equiv.) dropwise under a N₂ atmosphere. The reaction mixture was stirred at 0° C. for 30-35 minutes and consumption of the starting material was observed after 5-15 minutes. The reaction mixture was warmed to room temperature over 10-35 minutes. The reaction mixture was warmed to 50° C. and allowed to stir for approximately 20-23 hours under a N₂ atmosphere. The reaction was quenched with saturated aqueous NaHCO₃ (4 mL) and extracted with Et₂O. The organic layers were combined, washed with H₂O, brine and dried over Na₂SO₄. The mixture was filtered and solvent was removed in vacuo. The crude product was purified by distillation at 175-200° C. and 0.015-0.020 mmHg to yield the final product.

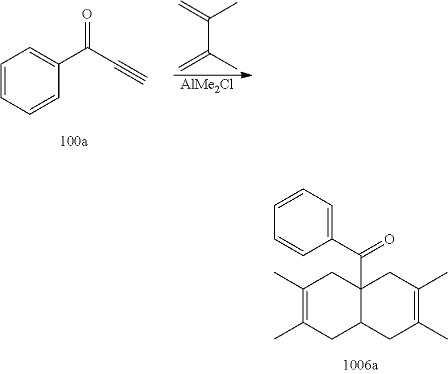

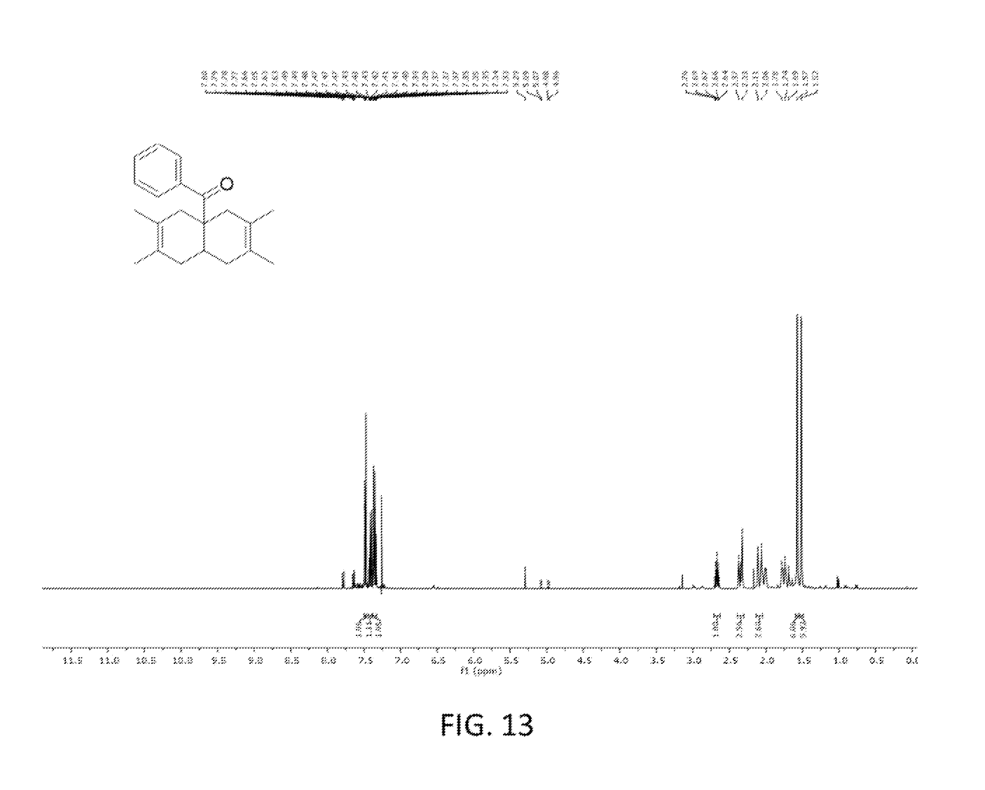

1006a was synthesized by adaptation of a known procedure¹: To a solution of 100a (201.7 mg, 1.56 mmol) and 2,3-dimethyl-1,3-butadiene (883 μL, 7.8 mmol) in dichloromethane (20 mL) at 0° C. was added AlMe₂Cl (1M, 1.56 mL, 1.56 mmol) dropwise under a N₂ atmosphere. The reaction mixture was stirred at 0° C. for 30 minutes and consumption of the starting material was observed after 5 minutes. The reaction mixture was warmed to room temperature and allowed to stir for 21.5 hours under a N₂ atmosphere. The reaction was quenched with saturated aqueous NaHCO₃ (8 mL) and extracted with CH₂Cl₂. The organic layers were combined, washed with H₂O, brine, and dried over Na₂SO₄. The mixture was filtered and solvent was removed in vacuo. The crude product was purified by distillation at 175° C. and 0.020 mmHg to yield 1006a (0.225 g, 73%) as a yellow oil. ¹H NMR (400 MHz, CDCl₃): δ 7.65-7.48 (m, 2H), 7.47-7.43 (m, 1H), 7.41-7.33 (m, 2H), 2.70-2.64(m, 1H), 2.37-2.06 (dd, 4H), 1.57 (s, 6H), 1.52 (s, 6H). See FIG. 13 for NMR spectrum.

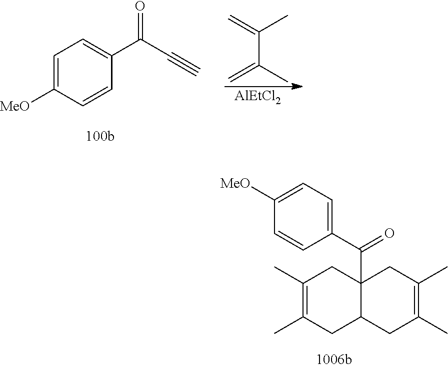

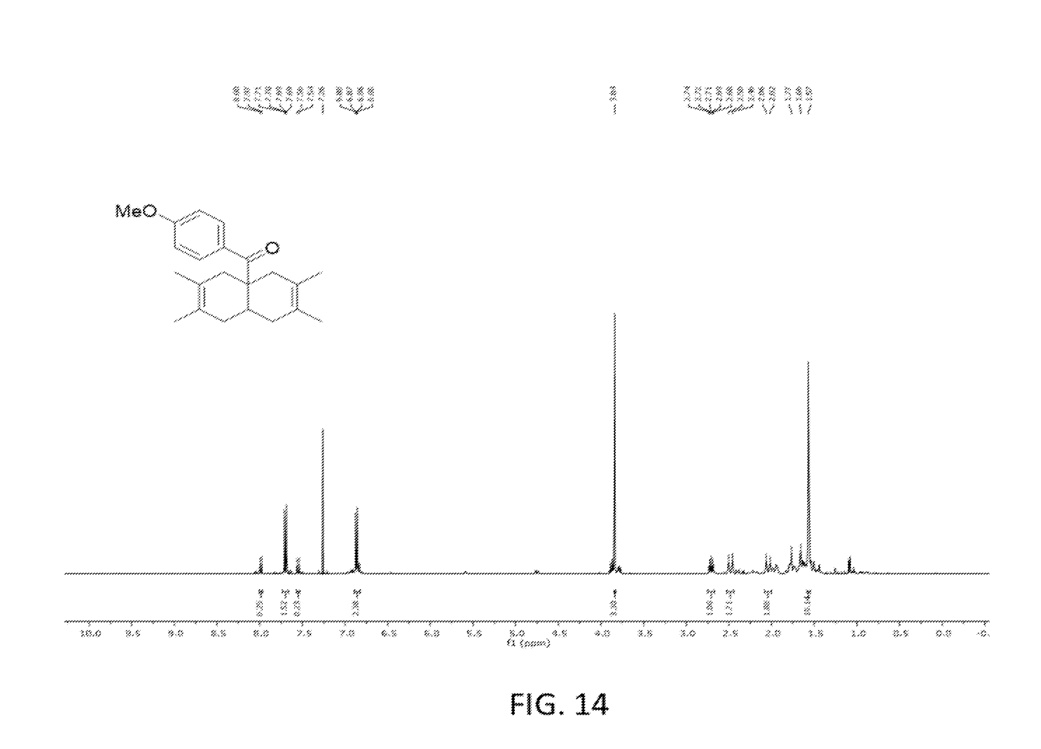

1006b: Performed according to the general procedure (0.63 mmol of 100b, 3.16 mmol of 2,3-dimethyl-1,3-butadiene, and 0.63 mmol AlEtCl₂). Purification by distillation at 200° C. and 0.020 mmHg to yield 1006b as yellow oil (121.8 mg, 59.4%). ¹H NMR (400 MHz, CDCl₃): δ 7.71-7.69 (m, 2H), 6.88-6.86 (m, 2H), 3.84 (s, 3H), 2.74-2.68 (m, 1H), 2.50-1.66 (dd, 4H), 1.57 (s, 12H). See FIG. 14 for NMR spectrum.

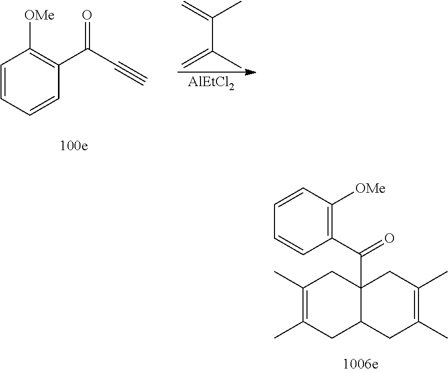

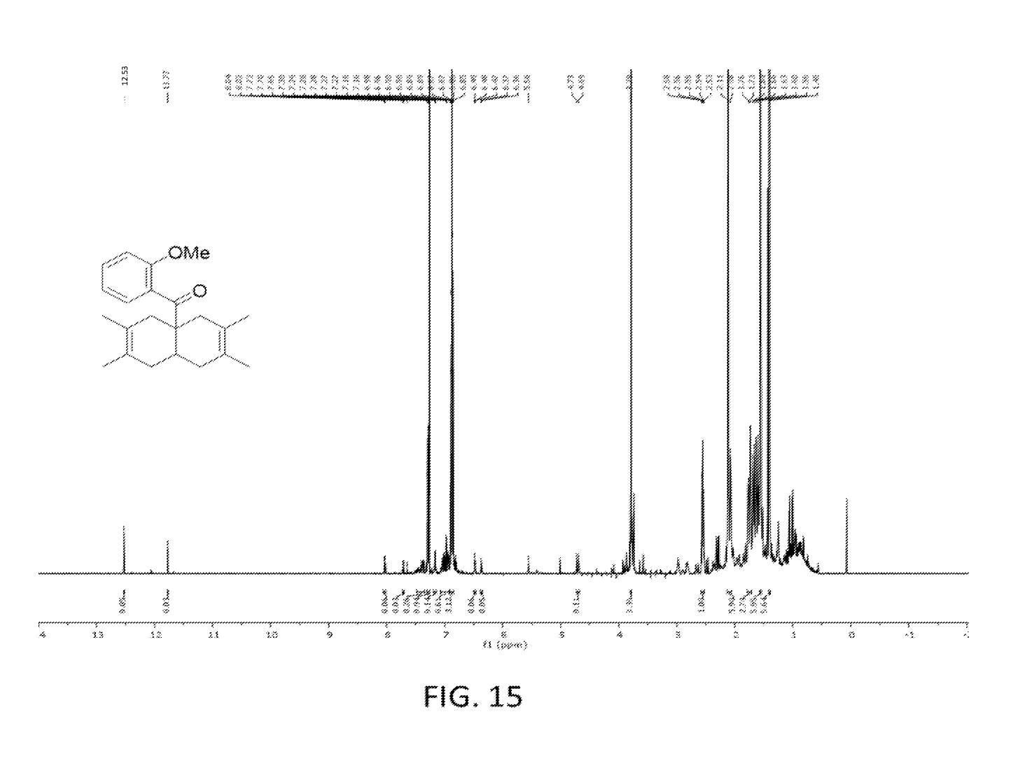

1006e: Performed according to the general procedure (0.68 mmol of 100e, 3.42 mmol of 2,3-dimethyl-1,3-butadiene, and 0.68 mmol AlEtCl₂). Purification by distillation at 175° C. and 0.020 mmHg to yield 1006e as yellow oil (0.110 g, 47.5%). ¹H NMR (500 MHz, CDCl₃): δ 6.96-6.90 (m, 1H), 6.89-6.86 (m, 4H), 3.70 (s, 3H), 2.58-2.53 (m, 1H), 2.11-1.73 (dd, 4H), 1.56 (s, 6H), 1.40 (s, 6H). See FIG. 15 for NMR spectrum.

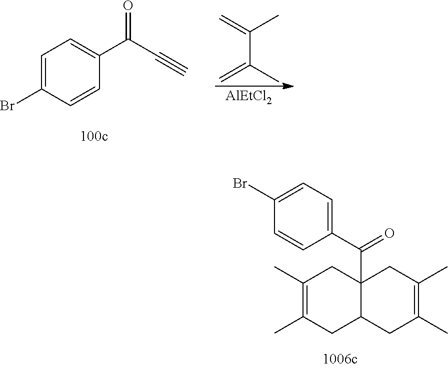

1006c: Performed according to the general procedure (0.48 mmol of 100c, 2.39 mmol of 2,3-dimethyl-1,3-butadiene, and 0.48 mmol AlEtCl₂). Purification by distillation to yield 1006c as yellow oil.

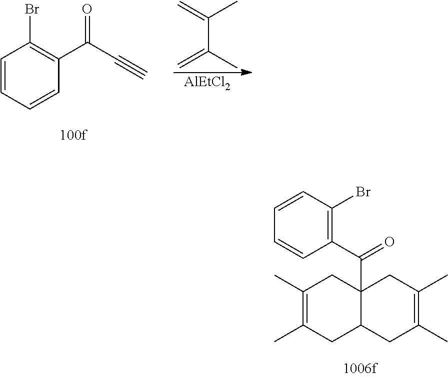

1006f: Performed according to the general procedure (0.48 mmol of 100f, 2.40 mmol of 2,3-dimethyl-1,3-butadiene, and 0.48 mmol AlEtCl₂). Purification by distillation to yield 1006f as yellow oil.

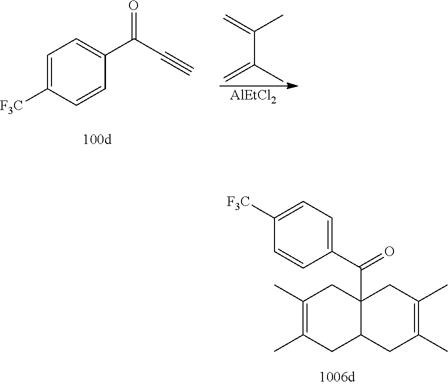

1006d: Performed according to the general procedure (0.51 mmol of 100d, 2.55 mmol of 2,3-dimethyl-1,3-butadiene, and 0.51 mmol AlEtCl₂). Purification by distillation to yield 1006d as yellow oil.

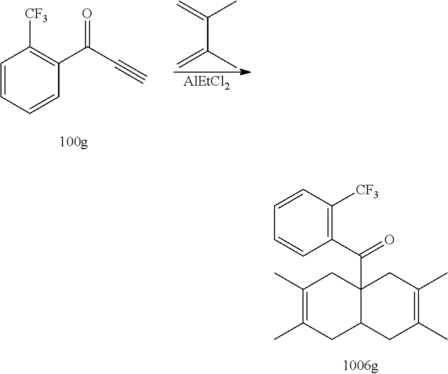

1006g: Performed according to the general procedure (0.55 mmol of 100g, 2.74 mmol of 2,3-dimethyl-1,3-butadiene, and 0.55 mmol AlEtCl₂). Purification by distillation to yield 1006g as yellow oil.

### IV. Overview of Several Embodiments

In some embodiments, a method, comprising combining an alkyne-containing compound comprising a carbonyl functional group, a diene, and a Lewis acid to form a multicyclic compound comprising one or more fused ring systems is described.

In any or all of the above embodiments, the alkyne-containing compound has a formula

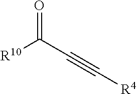

wherein R⁴ is selected from aryl, aliphatic, heteroaryl, heteroaliphatic, or —Si(R)₃, and R¹⁰ is selected from aryl, heteroaryl, aliphatic, ester, or carboxylic acid.

In any or all of the above embodiments, the alkyne-containing compound is a diynone.

In any or all of the above embodiments, the diynone comprises two terminal functional groups that facilitate regiochemical control.

In any or all of the above embodiments, the diynone has a formula

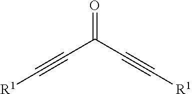

wherein each R¹ independently is a terminal functional group that facilitates regiochemical control.

In any or all of the above embodiments, each R¹ independently is selected from aryl, aliphatic, heteroaryl, heteroaliphatic, or —Si(R)₃, wherein each R independently is selected from hydrogen, aliphatic, heteroaliphatic, aryl, or heteroaryl.

In any or all of the above embodiments, each R¹ independently is selected from phenyl; pyridinyl; thiophenyl; naphthyl; meta-, ortho-, orpara-methoxyphenyl; meta-, ortho-, or para-trifluoromethylphenyl; meta-, ortho-, orpara-bromophenyl; meta-, ortho-, orpara-fluorophenyl; meta-, ortho-, orpara-chlorophenyl; meta-, ortho-, orpara-iodophenyl; cyclopropyl; cyclobutyl; cyclopentyl; cyclohexyl; methyl; ethyl; propyl; butyl; pentyl; or t-butyl.

In any or all of the above embodiments, the alkyne-containing compound has a formula

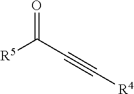

wherein R⁴ is selected from hydrogen, aliphatic, aryl, heteroaliphatic, or —Si(R)₃ wherein each R independently is selected from hydrogen, aliphatic, heteroaliphatic, aryl, or heteroaryl; and R⁵ is selected from aryl, heteroaryl, aliphatic, ester, or carboxylic acid.

In any or all of the above embodiments, R⁴ is selected from hydrogen, methyl, ethyl, propyl, butyl, pentyl, phenyl, methoxy, ethoxy, propoxy, —Si(Me)₃, —Si(Et)₃, or —Si(iPr)₃.

In any or all of the above embodiments, R⁵ is selected from phenyl; pyridinyl; meta-, ortho-, or para-methoxyphenyl; meta-, ortho-, or para-trifluoromethylphenyl; meta-, ortho-, orpara-bromophenyl; meta-, ortho-, or para-fluorophenyl; meta-, ortho-, or para-chlorophenyl; meta-, ortho-, or para-iodophenyl; methyl; ethyl; propyl; butyl; pentyl; —C(O)OH; or —C(O)OR, wherein R is selected from hydrogen, aliphatic, heteroaliphatic, aryl, and heteroaryl.

In any or all of the above embodiments, the diene has a formula

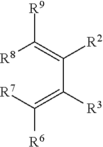

wherein R² is selected from hydrogen, aliphatic, heteroaliphatic, aryl, or heteroaryl; R³ is selected from hydrogen, aliphatic, heteroaliphatic, aryl, or heteroaryl; and each R⁶, R⁷, R⁸, and R⁹ independently is selected from hydrogen, aliphatic, heteroaliphatic, aryl, or heteroaryl.

In any or all of the above embodiments, each of R² and R³ independently is selected from hydrogen, methyl, ethyl, propyl, butyl, pentyl, —OSi(Me)₃, —OSi(Et)₃, or —OSi(iPr)₃; and each R⁶, R⁷, R⁸, and R⁹ independently is selected from hydrogen, methyl, ethyl, propyl, butyl, pentyl, methoxy, ethoxy, propoxy, —OSi(Me)₃, —OSi(Et)₃, —OSi(iPr)₃, phenyl, or pyridinyl.

In any or all of the above embodiments, the diene has a formula

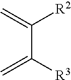

wherein R² is selected from hydrogen, aliphatic, heteroaliphatic, aryl, or heteroaryl; R³ is selected from hydrogen, aliphatic, heteroaliphatic, aryl, or heteroaryl.

In any or all of the above embodiments, the Lewis acid is selected from ZnCl₂, BF₃.Et₂O, SnCl₄, TiCl₄, FeCl₃, AlCl₃, EtAlCl₂, Me₂AlCl, BCl₃, or In(OTf)₃.

In any or all of the above embodiments, the method further comprises forming an intermediate having a structure

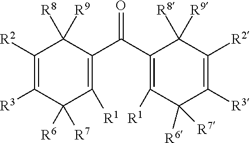

wherein each R¹ independently can be selected from aliphatic, aryl, heteroaryl, heteroaliphatic, or —Si(R)₃, wherein each R is selected from hydrogen, aliphatic, heteroaliphatic, aryl, heteroaryl; each R² and R^{2′} independently can be selected from hydrogen, aliphatic, heteroaliphatic, aryl, or heteroaryl; each R³ and R^{3′} independently can be selected from hydrogen, aliphatic, heteroaliphatic, aryl, or heteroaryl; and each R⁶, R⁷, R⁸, R⁹, R^{6′}, R^{7′}, R^{8′}, and R^{9′} independently can be selected from hydrogen, aliphatic, heteroaliphatic, aryl, or heteroaryl; or R⁷ or R^{7′} forms a 5-membered or 6-membered ring with R⁸ or R^{8′}, respectively.

In any or all of the above embodiments, the multicyclic compound has a formula

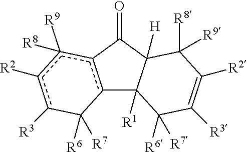

wherein each R¹ independently can be selected from aliphatic, aryl, heteroaryl, heteroaliphatic, or —Si(R)₃, wherein each R is selected from hydrogen, aliphatic, heteroaliphatic, aryl, heteroaryl; each R² and R^{2′} independently can be selected from hydrogen, aliphatic, heteroaliphatic, aryl, or heteroaryl; each R³ and R^{3′} independently can be selected from hydrogen, aliphatic, heteroaliphatic, aryl, or heteroaryl; and each R⁶, R⁷, R⁸, R⁹, R^{6′}, R^{7′}, R^{8′}, and R^{9′} independently can be selected from hydrogen, aliphatic, heteroaliphatic, aryl, or heteroaryl; or R⁷ or R^{7′} forms a 5-membered or 6-membered ring with R⁸ or R^{8′}, respectively.

In any or all of the above embodiments, the multicyclic compound has a structure selected from

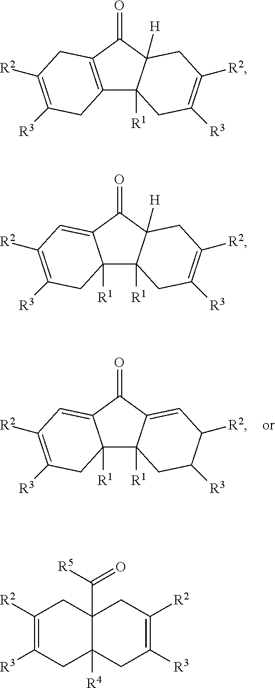

wherein each R¹ independently can be selected from aliphatic, aryl, heteroaryl, heteroaliphatic, or —Si(R)₃, wherein each R is selected from hydrogen, aliphatic, heteroaliphatic, aryl, heteroaryl; each R² independently is selected from hydrogen, aliphatic, heteroaliphatic, aryl, or heteroaryl; each R³ independently is selected from hydrogen, aliphatic, heteroaliphatic, aryl, or heteroaryl; R⁴ is selected from hydrogen, aliphatic, aryl, heteroaliphatic, or —OSi(R)₃; and R⁵ is selected from aryl, heteroaryl, aliphatic, ester, or carboxylic acid.

In view of the many possible embodiments to which the principles of the present disclosure may be applied, it should be recognized that the illustrated embodiments are only preferred examples of the disclosure and should not be taken as limiting the scope. Rather, the scope is defined by the following claims. We therefore claim as our invention all that comes within the scope and spirit of these claims.

1. A method, comprising combining an alkyne-containing compound comprising a carbonyl functional group, a diene, and a Lewis acid to form a multicyclic compound comprising one or more fused ring systems.

2. The method of claim 1, wherein the alkyne-containing compound has a formula 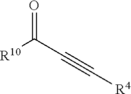
wherein R⁴ is selected from aryl, aliphatic, heteroaryl, heteroaliphatic, or —Si(R)₃, and R¹⁰ is selected from aryl, heteroaryl, aliphatic, ester, or carboxylic acid.

3. The method of claim 1, wherein the alkyne-containing compound is a diynone.

4. The method of claim 3, wherein the diynone comprises two terminal functional groups that facilitate regiochemical control.

5. The method of claim 3, wherein the diynone has a formula 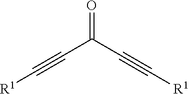
wherein each R¹ independently is a terminal functional group that facilitates regiochemical control.

6. The method of claim 5, wherein each R¹ independently is selected from aryl, aliphatic, heteroaryl, heteroaliphatic, or —Si(R)₃, wherein each R independently is selected from hydrogen, aliphatic, heteroaliphatic, aryl, or heteroaryl.

7. The method of claim 4, wherein each R¹ independently is selected from phenyl; pyridinyl; thiophenyl; naphthyl; meta-, ortho-, or para-methoxyphenyl; meta-, ortho-, or para-trifluoromethylphenyl; meta-, ortho-, or para-bromophenyl; meta-, ortho-, or para-fluorophenyl; meta-, ortho-, or para-chlorophenyl; meta-, ortho-, or para-iodophenyl; cyclopropyl; cyclobutyl; cyclopentyl; cyclohexyl; methyl; ethyl; propyl; butyl; pentyl; or t-butyl.

8. The method of claim 1, wherein the alkyne-containing compound is selected from

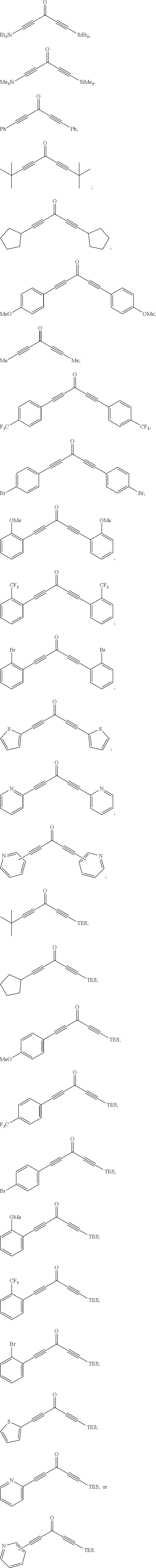

9. The method of claim 1, wherein the alkyne-containing compound has a formula 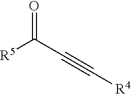
wherein R⁴ is selected from hydrogen, aliphatic, aryl, heteroaliphatic, or —Si(R)₃ wherein each R independently is selected from hydrogen, aliphatic, heteroaliphatic, aryl, or heteroaryl; and R⁵ is selected from aryl, heteroaryl, aliphatic, ester, or carboxylic acid.

10. The method of claim 9, wherein R⁴ is selected from hydrogen, methyl, ethyl, propyl, butyl, pentyl, phenyl, methoxy, ethoxy, propoxy, —Si(Me)₃, —Si(Et)₃, or —Si(iPr)₃.

11. The method of claim 9, wherein R⁵ is selected from phenyl; pyridinyl; meta-, ortho-, or para-methoxyphenyl; meta-, ortho-, or para-trifluoromethylphenyl; meta-, ortho-, or para-bromophenyl; meta-, ortho-, or para-fluorophenyl; meta-, ortho-, or para-chlorophenyl; meta-, ortho-, or para-iodophenyl; methyl; ethyl; propyl; butyl; pentyl; —C(O)OH; or —C(O)OR, wherein R is selected from hydrogen, aliphatic, heteroaliphatic, aryl, and heteroaryl.

12. The method of claim 1, wherein the alkyne-containing compound is selected from

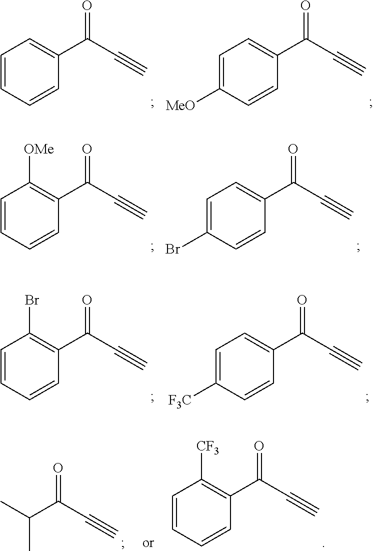

13. The method of claim 1, wherein the diene has a formula 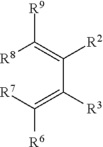
wherein R² is selected from hydrogen, aliphatic, heteroaliphatic, aryl, or heteroaryl; R³ is selected from hydrogen, aliphatic, heteroaliphatic, aryl, or heteroaryl; and each R⁶, R⁷, R⁸, and R⁹ independently is selected from hydrogen, aliphatic, heteroaliphatic, aryl, or heteroaryl.

14. The method of claim 13, wherein each of R² and R³ independently is selected from hydrogen, methyl, ethyl, propyl, butyl, pentyl, —OSi(Me)₃, —OSi(Et)₃, or —OSi(iPr)₃; and each R⁶, R⁷, R⁸, and R⁹ independently is selected from hydrogen, methyl, ethyl, propyl, butyl, pentyl, methoxy, ethoxy, propoxy, —OSi(Me)₃, —OSi(Et)₃, —OSi(iPr)₃, phenyl, or pyridinyl.

15. The method of claim 1, wherein the diene has a formula 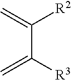
wherein R² is selected from hydrogen, aliphatic, heteroaliphatic, aryl, or heteroaryl; R³ is selected from hydrogen, aliphatic, heteroaliphatic, aryl, or heteroaryl.

16. The method of claim 1, wherein the diene is selected from

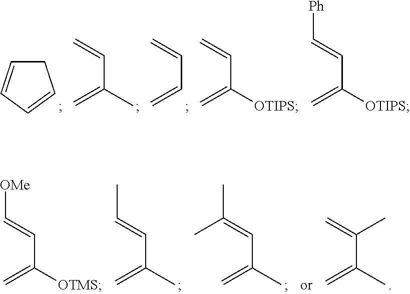

17. The method of claim 1, wherein the Lewis acid is selected from ZnCl₂, BF₃.Et₂O, SnCl₄, TiCl₄, FeCl₃, AlCl₃, EtAlCl₂, Me₂AlCl, BCl₃, or In(OTf)₃.

18. The method of claim 1, wherein the method further comprises forming an intermediate having a structure 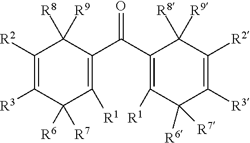
wherein each R¹ independently can be selected from aliphatic, aryl, heteroaryl, heteroaliphatic, or —Si(R)₃, wherein each R is selected from hydrogen, aliphatic, heteroaliphatic, aryl, heteroaryl; each R² and R^{2′} independently can be selected from hydrogen, aliphatic, heteroaliphatic, aryl, or heteroaryl; each R³ and R^{3′} independently can be selected from hydrogen, aliphatic, heteroaliphatic, aryl, or heteroaryl; and each R⁶, R⁷, R⁸, R⁹, R^{6′}, R^{7′}, R^{8′}, and R^{9′} independently can be selected from hydrogen, aliphatic, heteroaliphatic, aryl, or heteroaryl; or R⁷ or R^{7′} forms a 5-membered or 6-membered ring with R⁸ or R^{8′}, respectively.

19. The method of claim 1, wherein the multicyclic compound has a formula 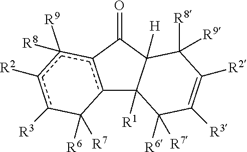
wherein each R¹ independently can be selected from aliphatic, aryl, heteroaryl, heteroaliphatic, or —Si(R)₃, wherein each R is selected from hydrogen, aliphatic, heteroaliphatic, aryl, heteroaryl; each R² and R^{2′} independently can be selected from hydrogen, aliphatic, heteroaliphatic, aryl, or heteroaryl; each R³ and R^{3′} independently can be selected from hydrogen, aliphatic, heteroaliphatic, aryl, or heteroaryl; and each R⁶, R⁷, R⁸, R⁹, R^{6′}, R^{7′}, R^{8′}, and R^{9′} independently can be selected from hydrogen, aliphatic, heteroaliphatic, aryl, or heteroaryl; or R⁷ or R^{7′} forms a 5-membered or 6-membered ring with R⁸ or R^{8′}, respectively.

20. The method of claim 1, wherein the multicyclic compound has a structure selected from 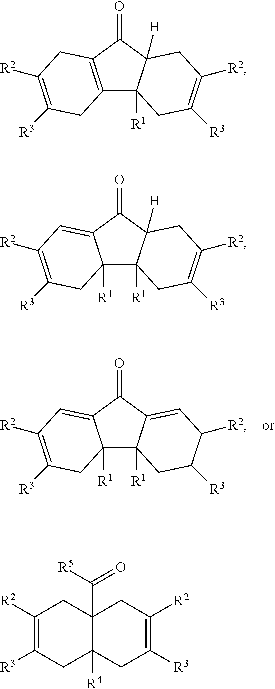
wherein each R¹ independently can be selected from aliphatic, aryl, heteroaryl, heteroaliphatic, or —Si(R)₃, wherein each R is selected from hydrogen, aliphatic, heteroaliphatic, aryl, heteroaryl; each R² independently is selected from hydrogen, aliphatic, heteroaliphatic, aryl, or heteroaryl; each R³ independently is selected from hydrogen, aliphatic, heteroaliphatic, aryl, or heteroaryl; R⁴ is selected from hydrogen, aliphatic, aryl, heteroaliphatic, or —OSi(R)₃; and R⁵ is selected from aryl, heteroaryl, aliphatic, ester, or carboxylic acid.

21. The method of claim 1, wherein the multicyclic compound is selected from:

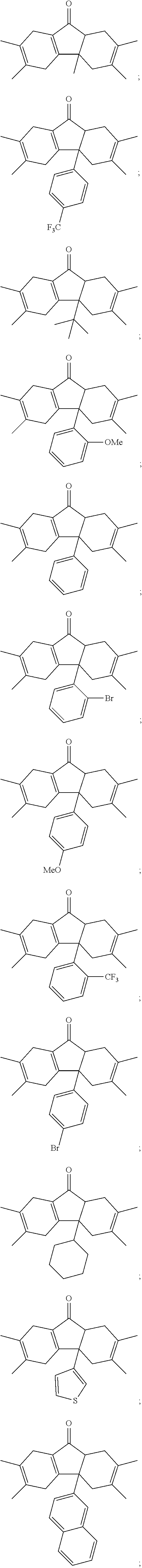

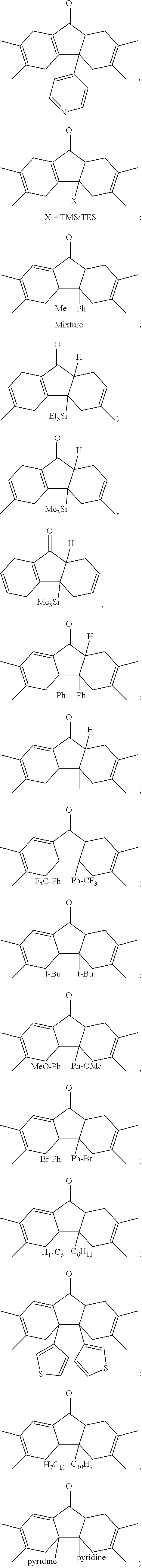

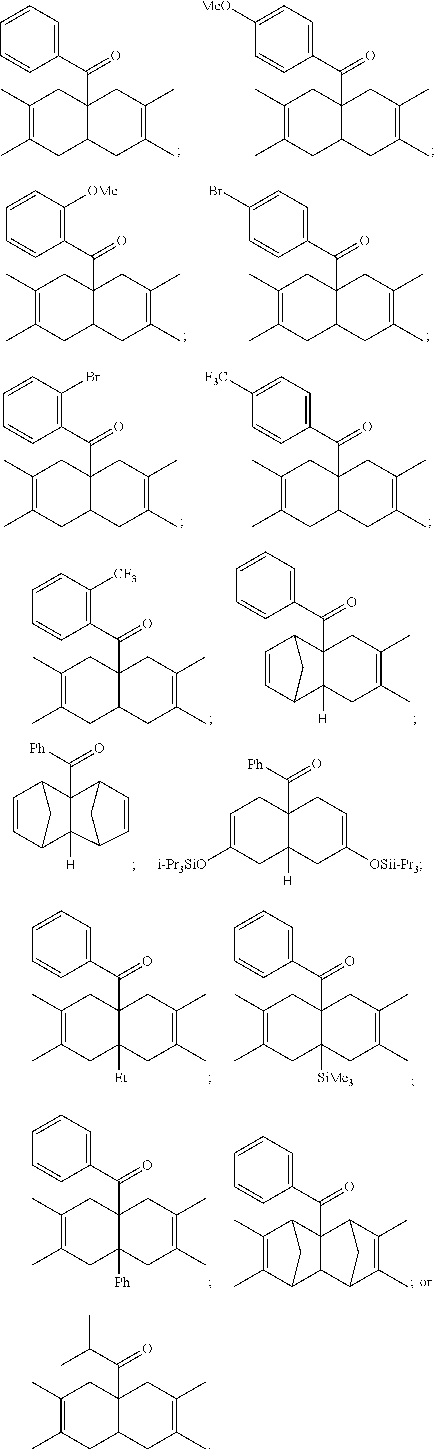

22. A compound selected from: 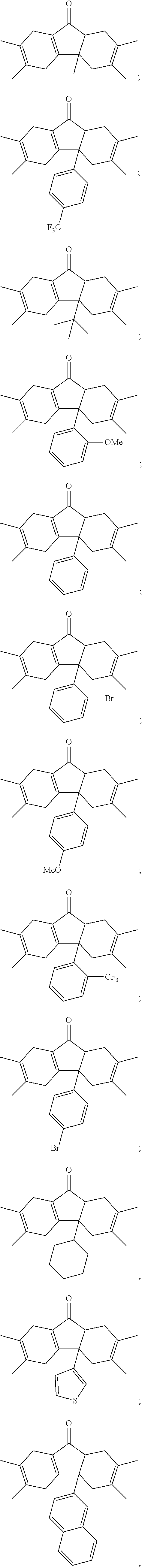 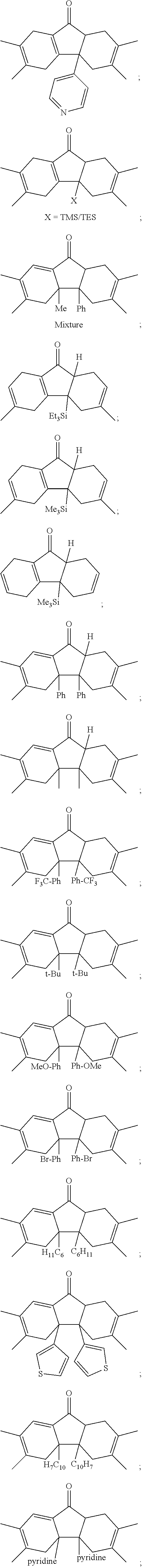 [chemical structure]
